SEQUENCE LISTING (23 sequences)

SEQ ID NO: 1 - protein, 108 aa, Artificial Sequence
  Synthetic
MATRTPKLVKHTLLTRFQDCITREQIDNYINDYTNLLDLIPSMQSFNWGTDLGMESAELNRGYTHAFESTFESKSGLQEYLDSAALAAFAEGFLPTLSQRLVIDYFLY

SEQ ID NO: 2 - DNA, 327 bp, Artificial Sequence
  Synthetic
atggcgacccgcaccccgaaactggttaaacacaccctgctgacccgcttccaggattgcattacccgcgaacagatcgacaactacatcaacgactacaccaacctgctggatctgattccgagcatgcagagcttcaactggggcaccgacctgggtatggagagcgcggaactgaaccgtggttacacccacgcgttcgagagcacctttgaaagcaaaagcggcctgcaggagtatctggatagcgcggcgctggcggcgtttgcggaaggttttctgccgaccctgagccaacgcctggttattgattactttctgtattaa

SEQ ID NO: 3 - DNA, 10 bp, Artificial Sequence
  Synthetic
  misc_feature (1)..(1): TCO
ctggaactgt

SEQ ID NO: 4 - DNA, 10 bp, Artificial Sequence
  Synthetic
  misc_feature (1)..(1): TCO-Cy5
ctggaactgt

SEQ ID NO: 5 - DNA, 10 bp, Artificial Sequence
  Synthetic
  misc_feature (1)..(1): TCO
acagttccag

SEQ ID NO: 6 - DNA, 10 bp, Artificial Sequence
  Synthetic
  misc_feature (1)..(1): TCO- Cy3
acagttccag

SEQ ID NO: 7 - DNA, 8 bp, Artificial Sequence
  Synthetic
  misc_feature (1)..(1): TCO
aatatatt

SEQ ID NO: 8 - DNA, 8 bp, Artificial Sequence
  Synthetic
  misc_feature (1)..(1): TCO-Cy5
aatatatt

SEQ ID NO: 9 - DNA, 8 bp, Artificial Sequence
  Synthetic
  misc_feature (1)..(1): TCO
  misc_feature (8)..(8): Cy3
aatatatt

SEQ ID NO: 10 - DNA, 6 bp, Artificial Sequence
  Synthetic
  misc_feature (1)..(1): TCO
tttttt

SEQ ID NO: 11 - DNA, 6 bp, Artificial Sequence
  Synthetic
  misc_feature (1)..(1): TCO-Cy5
tttttt

SEQ ID NO: 12 - DNA, 6 bp, Artificial Sequence
  Synthetic
  misc_feature (1)..(1): TCO
  misc_feature (6)..(6): Cy3
tttttt

SEQ ID NO: 13 - DNA, 10 bp, Artificial Sequence
  Synthetic
  misc_feature (1)..(1): DBCO
ctacaaatct

SEQ ID NO: 14 - DNA, 10 bp, Artificial Sequence
  Synthetic
  misc_feature (1)..(1): DBCO-Cy3
ctacaaatct

SEQ ID NO: 15 - DNA, 10 bp, Artificial Sequence
  Synthetic
  misc_feature (1)..(1): DBCO
agatttgtag

SEQ ID NO: 16 - DNA, 10 bp, Artificial Sequence
  Synthetic
  misc_feature (1)..(1): DBCO-Cy5
agatttgtag

SEQ ID NO: 17 - DNA, 6 bp, Artificial Sequence
  Synthetic
  misc_feature (1)..(1): DBCO
aatatt

SEQ ID NO: 18 - DNA, 6 bp, Artificial Sequence
  Synthetic
  misc_feature (1)..(1): DBCO-Cy3
aatatt

SEQ ID NO: 19 - DNA, 6 bp, Artificial Sequence
  Synthetic
  misc_feature (1)..(1): DBCO-Cy5
aatatt

SEQ ID NO: 20 - DNA, 6 bp, Artificial Sequence
  Synthetic
  misc_feature (1)..(1): DBCO
taatta

SEQ ID NO: 21 - DNA, 8 bp, Artificial Sequence
  Synthetic
  misc_feature (1)..(1): DBCO
tttttttt

SEQ ID NO: 22 - DNA, 8 bp, Artificial Sequence
  Synthetic
  misc_feature (1)..(1): DBCO-Cy3
tttttttt

SEQ ID NO: 23 - DNA, 8 bp, Artificial Sequence
  Synthetic
  misc_feature (1)..(1): DBCO-Cy5
tttttttt

(19) United States
(12) Patent Application Publication
(10) Pub. No.: US 2022/0372062 A1
(43) Pub. Date: Nov. 24, 2022

(54) ENCODING HIERARCHICAL ASSEMBLY PATHWAYS OF PROTEINS WITH DNA

(71) Applicant: NORTHWESTERN UNIVERSITY, Evanston, IL (US)

(72) Inventors: Chad A. Mirkin, Wilmette, IL (US); Oliver G. Hayes, Evanston, IL (US); Benjamin Edward Partridge, Chicago, IL (US)

(21) Appl. No.: 17/752,407

(22) Filed: May 24, 2022

Related U.S. Application Data
(60) Provisional application No. 63/192,276, filed on May 24, 2021.

(51) Int. Cl. C07H 21/00

(52) U.S. Cl. CPC C07H 21/00

(57) ABSTRACT

Provided herein are hierarchical protein structures comprising two or more proteins extending in one or more dimensions, the hierarchical protein structure comprising: a first protein comprising: (i) a patch A comprising one or more polynucleotides conjugated to the surface of the first protein; and (ii) a patch B comprising one or more polynucleotides conjugated to the surface of the first protein; and a second protein comprising: (i) a patch A′ comprising one or more polynucleotides conjugated to the surface of the second protein; and (ii) a patch B′ comprising one or more polynucleotides conjugated to the surface of the second protein; wherein the one or more polynucleotides of the patch A hybridizes to the one or more polynucleotides of the patch A′, and/or the one or more polynucleotides of the patch B hybridizes to the one or more polynucleotides of the patch B′ to form the hierarchical protein structure. Also provided are methods of making the hierarchical protein structures.

### CROSS-REFERENCE TO RELATED APPLICATIONS

This application claims the priority benefit under 35 U.S.C. § 119(e) of U.S. Provisional Patent Application No. 63/192,276, filed May 24, 2021, which is incorporated herein by reference in its entirety.

### STATEMENT OF GOVERNMENT SUPPORT

This invention was made with government support under FA9550-16-1-0150, awarded by the Air Force Office of Scientific Research (AFOSR), and N00014-16-1-3117 awarded by The Office of Naval Research (ONR). The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2021-094_Seqlisting.txt”, which was created on May 23, 2022 and is 7,107 bytes in size. The subject matter of the Sequence Listing is incorporated herein in its entirety by reference.

### BACKGROUND

Hierarchical assembly is integral to the structural complexity and function of materials and systems that occur in Nature. Muscle tissue, amyloid fibrils, and collagen networks are all examples of highly organized supramolecular architectures that arise from bottom-up, multi-step, regulated assembly processes. The well-controlled sequence of assembly steps along a given pathway and the specificity of interactions between components are critical to the observed structural complexity and diversity. While nanoscale hierarchical assembly is prevalent and important in Nature, and the ability to control the bottom-up assembly of synthetic nanoscale building blocks has been transformed over the past two decades, the ability to program through hierarchical mechanisms remains limited. This is due to difficulties in defining the number, type, and location of multiple interactions on synthetic building blocks, as well as limitations in controlling the interplay between orthogonal interactions to achieve a desired assembly pathway.

### SUMMARY

The development of tools and strategies to program multi-step assembly pathways of nanoscale building blocks would redefine how to control the bottom-up synthesis of materials and accelerate the discovery of novel structures with desirable properties and functions. Described herein are methods for addressing this gap by spatially encoding programmable interacting ligands (DNA) onto the surface of chemically addressable building blocks (proteins).

Provided herein are comprising two or more proteins extending in one or more dimensions, the hierarchical protein structure comprising: a first protein comprising: (i) a patch A comprising one or more polynucleotides conjugated to the surface of the first protein; and (ii) a patch B comprising one or more polynucleotides conjugated to the surface of the first protein; and a second protein comprising: (i) a patch A′ comprising one or more polynucleotides conjugated to the surface of the second protein; and (ii) a patch B′ comprising one or more polynucleotides conjugated to the surface of the second protein; wherein the one or more polynucleotides of the patch A hybridizes to the one or more polynucleotides of the patch A′, and/or the one or more polynucleotides of the patch B hybridizes to the one or more polynucleotides of the patch B′ to form the hierarchical protein structure. Also provided are hierarchical protein structures wherein the one or more polynucleotides of the patch A hybridizes to the one or more polynucleotides of the patch A′, and the one or more polynucleotides of the patch B hybridizes to the one or more polynucleotides of the patch B′ to form the hierarchical protein structure.

Also provided are methods of making the hierarchical protein structures disclosed herein, comprising contacting: (a) a first protein comprising: (i) a patch A comprising one or more polynucleotides conjugated to the surface of the first protein; and (ii) a patch B comprising one or more polynucleotides conjugated to the surface of the first protein; and (b) a second protein comprising: (i) a patch A′ comprising one or more polynucleotides conjugated to the surface of the second protein; and (ii) a patch B′ comprising one or more polynucleotides conjugated to the surface of the second protein; wherein the one or more polynucleotides of the patch A is sufficiently complementary to the one or more polynucleotides of the patch A′ to hybridize, and wherein the contacting is performed under conditions that result in the one or more polynucleotides of the patch A hybridizing to the one or more polynucleotides of the patch A′, thereby making the hierarchical protein structure.

Also provided are methods wherein the one or more polynucleotides of the patch B is sufficiently complementary to the one or more polynucleotides of the patch B′ to hybridize under said conditions. Further provided are methods further comprising hybridizing the one or more polynucleotides of the patch B to the one or more polynucleotides of the patch B′, thereby making the hierarchical protein structure extending in a second dimension.

### BRIEF DESCRIPTION OF THE FIGURES

FIG. 1 shows the design of Sp1m chemical surface and proposed hierarchical assembly schemes. (A) Native Sp1 (left) presents multiple primary amines (lysines and N-termini, darker residues) and no cysteines on its surface. Three mutations were designed to remove two native lysines and introduce one cysteine. Due to the dodecameric structure of Sp1m, these mutations (right) define the chemical anisotropy across the protein surface with amine residues only on the axial face and cysteines (darker residues at the 2, 4, 6, 8, 10, and 12 o'clock positions of top right structure, and left, middle, and right of bottom right structure) located only on the equatorial face. (B) Proposed assembly schemes for building blocks containing strong or weak surface interactions at their axial or equatorial positions. Without wishing to be bound by theory, strong interactions direct the first stage of assembly, leading to multivalency among weak interactions that direct the second stage of assembly.

FIG. 2 shows the synthesis and characterization of Sp1m-DNA conjugates. (A) Sp1m (1) was modified with DNA in three steps: (i) cysteines were first modified with Linker 1 (C) through a thiol-maleimide Michael addition click reaction to give Sp1m-N₃ (2); (ii) primary amines were then modified with Linker 2 (C) to generate 3 through reaction with an NHS-activated ester; (iii) TCO- and DBCO-modified DNA were reacted with 3 in one-pot to generate a Sp1m-DNA building block (4). (B) Negative stain TEM of (1). Scale bar is 50 nm. Lower image: comparison of a model of Sp1m with a magnified region from the TEM image. (C) Chemical structures of heterobifunctional Linkers 1 and 2. (D) MALDI-TOF MS confirming the consecutive addition of a single molecule of each linker to each subunit of 1. (E) Denaturing PAGE (left to right) protein ladder, unreacted Sp1m (1), and purified Sp1m-DNA conjugate (4). The presence of two bands of approximately equal intensity, at higher molecular weight compared to 1, correspond to a roughly equal mixture of protein subunits with 1 and 2 DNA strands.

FIG. 3 shows the design of Sp1m building block and hierarchical assembly pathways. (A) Proposed assembly schemes for building blocks containing strong or weak surface interactions at their axial or equatorial positions. Without wishing to be bound by theory, strong interactions direct the first stage of assembly, leading to multivalency among weak interactions that direct the second stage of assembly. (B) Hierarchical assembly of Sp1m-A_{S}E_{W1} and Sp1m-A′_{S}E_{W2} building blocks showing negative stain TEM characterization of structures after each stage of assembly. Scale bars are 150 nm.

FIG. 4 shows the characterization of the assembly of Sp1m with strong axial (A_{S}/A′_{S}) interactions. (A) Scheme showing the donor-quenching FRET experiment. In a typical experiment, a pair of complementary Sp1m-DNA conjugates were functionalized with Cy3- or Cy5-modified axial DNA, respectively. When well separated, excitation of Cy3 results in fluorescence from Cy3 (filled circle). However, when Cy3 and Cy5 are in close proximity, FRET from excited Cy3 to Cy5 quenches the fluorescence of Cy3 leading to reduced fluorescent signal (empty circle). (B) Temperature-dependent association of Sp1m-A_{S}E_{NC} and Sp1m-A′_{S}E_{NC} represented as fraction assembled vs temperature, where the fluorescence intensities at 65 and 20° C. correspond to a fraction assembled of 0 and 1, respectively. (C) Negative stain and (D) cryogenic TEM micrographs of slow cooled Sp1m-A_{S}E_{NC} and Sp1m-A′_{S}E_{NC}. Scale bars are 150 nm.

FIG. 5 shows the characterization of the assembly of Sp1m with strong equatorial (E_{S}/E′_{S}) interactions. (A) Schematic of the donor-quenching FRET experiment. (B) Temperature-dependent association of Sp1m-E_{S} and Sp1m-E′_{S} represented by plot of fraction assembled vs temperature. (C) Negative stain TEM micrograph of slow-cooled Sp1m-E_{S} and Sp1m-E′_{S}. Scale bar is 150 nm. (D) Liquid AFM micrograph of slow-cooled Sp1m-E_{S} and Sp1m-E′_{S}. White arrow denotes line used for height profile in (E).

FIG. 6 shows the FRET-based characterization of temperature-dependent hierarchical assembly processes. (A-C) Hierarchical assembly mediated by strong axial (A_{S}/A′_{S}) interactions. (A) Scheme showing the hypothesized assembly outcomes for two pairs of A_{S}/A′_{S} building blocks: Sp1m-A_{S}E_{W1} with Sp1m-A′_{S}E_{W1}; and Sp1m-A_{S}E_{NC} with Sp1m-A′_{S}E_{NC}. Temperature-dependent association of (B) Sp1m-A_{S}E_{W1} and Sp1m-A′_{S}E_{W1} and (C) Sp1m-A_{S}E_{NC} and Sp1m-A′_{S}E_{NC} represented by plots of fraction assembled vs temperature. Both pairs show the first stage of assembly mediated by A_{S}/A′_{S} interactions, but only with E_{W1} is a second stage of assembly observed. (D-F) Hierarchical assembly mediated by strong equatorial (E_{S}/E′_{S}) interactions. (D) Scheme showing hypothesized assembly outcomes for two pairs of E_{S}/E′_{S} building blocks: Sp1m-A_{W}E_{S} with Sp1m-A_{W}E′_{S}; and Sp1m-A_{NC}E_{S} with Sp1m-A_{NC}E′_{S}. Temperature-dependent association of (E) Sp1m-A_{W}E_{S} and Sp1m-A_{W}E′_{S} and (F) Sp1m-A_{NC}E_{S} and Sp1m-A_{NC}E′_{S} represented by plots of fraction assembled vs temperature. Both pairs show the first stage of assembly mediated by E_{S}/E′_{S} interactions, but only with A_{W} is a second stage of assembly observed.

FIG. 7 shows the characterization of assembly outcomes from axial-first, equatorial-second hierarchical assembly processes. (A) Scheme showing 1D protein chains displaying equatorial E_{W1} DNA homogenously. (B) Negative-stain TEM micrograph of slow-cooled assembly of Sp1m-A_{S}E_{W1} and Sp1m-A′_{S}E_{W1}. (C) Scheme showing 1D protein chains displaying alternating equatorial E_{W1} and Ewe DNA. (D) Negative-stain TEM micrograph of slow-cooled assembly of Sp1m-A_{S}E_{W1} and Sp1m-A′_{S}E_{W2}. Scale bars are 150 nm.

FIG. 8 shows the functionalization of Sp1m with azide and tetrazine Linkers.

FIG. 9 shows the observed and theoretical masses and corresponding linker attachment positions on Sp1m-2L.

FIG. 10 shows the local chemical environment of K74 (dark residue) showing hydrogen bonds (depicted as dashed lines) with adjacent amino acid residues and water molecules (labeled “H₂O”). Protein coordinates taken from PDB: 1TR0.

FIG. 11 shows representative negative-stain TEM micrographs of slow-cooled Sp1m-A_{S}E_{NC}. Scale bars are 150 nm.

FIG. 12 shows representative negative-stain TEM micrographs of slow-cooled Sp1m-E_{S}. (A) Micrograph showing a wide-field image of the sample. (B) An expanded view of outlined area in (A). Scale bars are 150 nm.

FIG. 13 shows graphs showing the influence of salt concentration on the assembly of Sp1m-A_{S}E_{W} and Sp1m-A′_{S}E_{W}. (A) Raw Cy3 intensity data of dye-labelled building blocks measured at different salt concentrations. (B) Fraction assembled vs temperature data normalized to greatest fraction assembled (20 mM MgCl₂) to show relative fraction assembled as a function of salt concentration.

FIG. 14 shows (A and B) AFM micrographs of slow-cooled Sp1m-A_{W}E_{S} and Sp1m-A_{W}E′_{S} reveal large area two-dimensional protein assembly containing areas of different heights. (C) Height profiles measured along lines 1-6 indicated in (A) revealing quantized layer heights, with increments measuring 6 nm.

FIG. 15 shows (A and B) Representative negative-stain TEM micrographs of slow-cooled Sp1m-A_{S}E_{W1} and Sp1m-A′_{S}E_{W1}. (C and D) Representative negative-stain TEM micrographs of slow-cooled Sp1m-A_{S}E_{W1} and Sp1m-A′_{S}E_{W2}. Scale bars are 150 nm.

### DETAILED DESCRIPTION

Proteins are an important class of nanoscale building block because of their structural and functional roles in biology. As such, developing methods to synthetically engineer new materials from proteins is a common goal in the fields of synthetic biology, chemistry, and materials science. The chemical complexity of protein surfaces defines specific recognition between protein interfaces and is key to the hierarchical assembly processes observed in Nature. However, their complex surfaces make it challenging to design protein building blocks that will transform into targeted materials by traversing an intended assembly pathway. While powerful de novo design strategies have been utilized to create proteins with predetermined interfaces and assembly outcomes, this approach inherently deviates from the pool of naturally occurring protein building blocks that could be utilized for materials engineering. Other strategies have relied on introducing controlled molecular interactions to the surfaces of proteins ranging from metal coordination chemistries to hydrophobic and host-guest interactions. However, achieving specificity and orthogonality through these means can be challenging. Despite significant innovation in manipulating surface interactions through chemical modifications, less attention has been paid to designing protein building blocks that can undergo multi-step assembly pathways mimicking those in Nature. Methods to define interaction location and type on the surface of a building block, in conjunction with an understanding of how to control and regulate each interaction independently, are needed to successfully program hierarchical assembly pathways.

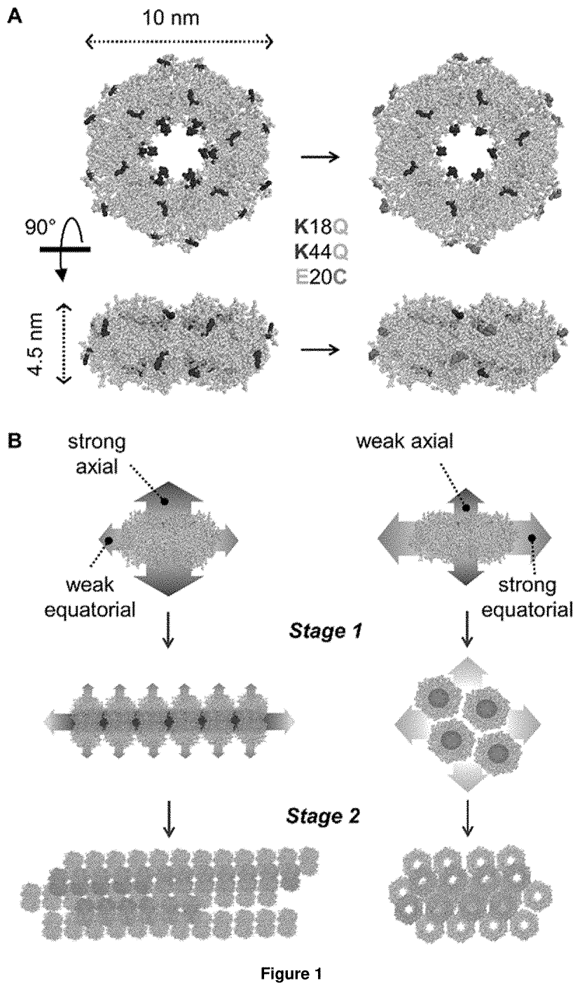

DNA ligands can be chemically tethered to the surfaces of proteins, at specific locations, to drive the assembly of proteins into one- and three-dimensional structures and crystals. Protein mutagenesis has been used to site-specifically encode multiple, orthogonal DNA interactions onto protein surfaces to program directional assembly. Furthermore, the programmable recognition properties of DNA surface ligands have been utilized to control the polymerization pathway of proteins. Defining the specificity, strength, and spatial distribution of multiple specific DNA interactions on the surface of a protein is a promising strategy for synthesizing protein building blocks that undergo programmed, multi-step assembly processes. Here, by defining the chemical anisotropy of a protein's surface via mutagenesis, DNA interactions can be defined spatially, that is, axially or equatorially with respect to the geometry of an anisotropic protein (FIG. 1A). Without wishing to be bound by theory, through careful DNA design, the relative interaction strengths of the axial and equatorial faces can be modulated to confine each assembly step to a single direction, thereby directing proteins to assemble hierarchically along specific, multi-step pathways (FIG. 1B).

This work harnesses the programmability of DNA and the chemical addressability of protein surfaces to control the hierarchical, multi-step assembly of protein building blocks mediated by multiple, distinct DNA hybridization events. Through functionalization of a protein's surface with DNA ligands at axial and equatorial positions, highly directional interactions are introduced between specific geometric interfaces. Multi-step assembly profiles can be programmed by defining disparate recognition properties at different locations within discrete protein building blocks, which allows for controlling the assembly pathways and structural outcomes. Furthermore, DNA can be used to define multiple orthogonal interactions within a single assembly pathway, thereby realizing distinct, novel protein-based materials as a function of both the type of pathway traversed and the DNA design employed. This principle, in which all information required for hierarchical assembly is encoded into an initial primary structure, has long been exploited by Nature to realize sophisticated architectures from amino acid sequences, but seldom by using nucleic acids. In contrast to canonical uses of nucleic acids in Nature—primarily information storage and sometimes as a template to organize structures—DNA is rarely, if ever, employed as a programmable “bond” to direct complex assembly pathways. These findings show that, through judicious design, one can use DNA to build structures on demand with a degree of hierarchical control atypical for synthetic nanoscale programmable matter but reminiscent of complex structures in Nature. These insights reveal how to go beyond a single-step assembly pathway for the bottom-up assembly of nanomaterials and will enable the synthesis of novel, hierarchically structured materials by design.

Provided herein are hierarchical protein structures comprising two or more proteins extending in one or more dimensions, the hierarchical protein structure comprising:

a first protein comprising: (i) a patch A comprising one or more polynucleotides conjugated to the surface of the first protein; and (ii) a patch B comprising one or more polynucleotides conjugated to the surface of the first protein; and

a second protein comprising: (i) a patch A′ comprising one or more polynucleotides conjugated to the surface of the second protein; and (ii) a patch B′ comprising one or more polynucleotides conjugated to the surface of the second protein;

wherein the one or more polynucleotides of the patch A hybridizes to the one or more polynucleotides of the patch A′, and/or the one or more polynucleotides of the patch B hybridizes to the one or more polynucleotides of the patch B′ to form the hierarchical protein structure. In some cases, the one or more polynucleotides of the patch A hybridizes to the one or more polynucleotides of the patch A′, and the one or more polynucleotides of the patch B hybridizes to the one or more polynucleotides of the patch B′ to form the hierarchical protein structure. As used herein, a “plurality of polynucleotides” comprises one or more polynucleotides.

As used herein, the term “hierarchical protein structure” refers to a self-assembled array of proteins in one, two, or three dimensions, wherein individual proteins are first assembled into ordered secondary structures via noncovalent interactions, which further act as building blocks in a further assembly step to form more complex superstructures at the next level via the formation of ordered tertiary or higher level structures via further noncovalent interactions.

### Proteins of the Disclosure

As used herein, the term “protein” refers to a polymer comprised of amino acid residues. Proteins are understood in the art and include without limitation antibodies, enzymes, structural proteins, and hormones. Thus, proteins contemplated by the disclosure include without limitation those having structural, catalytic, signaling, therapeutic, or transport activity.

Proteins of the present disclosure may be either naturally occurring or non-naturally occurring. Naturally occurring proteins include without limitation biologically active proteins (including antibodies) that exist in nature or can be produced in a form that is found in nature by, for example, chemical synthesis or recombinant expression techniques. Naturally occurring proteins also include lipoproteins and post-translationally modified proteins, such as, for example and without limitation, glycosylated proteins. Antibodies contemplated for use in the methods and compositions of the present disclosure include without limitation antibodies that recognize and associate with a target molecule either in vivo or in vitro. Structural proteins contemplated by the disclosure include without limitation actin, tubulin, collagen, elastin, myosin, kinesin and dynein.

Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “peptide” typically refers to short polypeptides/proteins.

Non-naturally occurring proteins are prepared, for example, using an automated protein synthesizer or, alternatively, using recombinant expression techniques using a modified polynucleotide which encodes the desired protein.

Fusion proteins, including fusion proteins wherein one fusion component is a fragment or a mimetic, are also contemplated. A “mimetic” as used herein means a peptide or protein having a biological activity that is comparable to the protein of which it is a mimetic. By way of example, an endothelial growth factor mimetic is a peptide or protein that has a biological activity comparable to the native endothelial growth factor. The term further includes peptides or proteins that indirectly mimic the activity of a protein of interest, such as by potentiating the effects of the natural ligand of the protein of interest.

### Polynucleotides of the Disclosure

Polynucleotides contemplated by the present disclosure include DNA, RNA, modified forms and combinations thereof as defined herein. Accordingly, in any of the aspects or embodiments of the disclosure, the hierarchical protein structures comprise DNA. In any of the aspects or embodiments of the disclosure, each polynucleotide that is part of a hierarchical protein structure is DNA. In any of the aspects or embodiments of the disclosure, each polynucleotide that is part of a hierarchical protein structure is RNA. In any of the aspects or embodiments of the disclosure, each polynucleotide that is part of a hierarchical protein structure is a modified polynucleotide. In some embodiments, the polynucleotides that are part of a hierarchical protein structure contain any combination of DNA, RNA, and/or modified polynucleotides. In any of the aspects or embodiments of the disclosure, the DNA is single-stranded. In some embodiments, the DNA is double stranded. Single stranded DNA also includes DNA with secondary structure, such as, for example and without limitation, G-quadruplexes and i-motifs. In further aspects, the hierarchical protein structures comprise RNA, and in still further aspects the hierarchical protein structures comprise double stranded RNA. The term “RNA” includes duplexes of two separate strands, as well as single stranded structures. Single stranded RNA also includes RNA with secondary structure. In one aspect, RNA having a hairpin loop is contemplated.

A “polynucleotide” is understood in the art to comprise individually polymerized nucleotide subunits. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases adenine (A), guanine (G), cytosine (C), thymine (T) and uracil (U). Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

A polynucleotide of the disclosure, or a modified form thereof, is generally from about 3 nucleotides to about 50 nucleotides in length. In general, the length of the polynucleotide will depend on protein size and where in the nucleotide sequence the polynucleotide is attached to the protein. More specifically, a polynucleotide can be about 2 to about 40 nucleotides in length, about 2 to about 30 nucleotides in length, about 2 to about 20 nucleotides in length, about 2 to about 10 nucleotides in length, or about 2 to about 5 nucleotides in length, and all polynucleotides intermediate in length of the sizes specifically disclosed to the extent that the polynucleotide is able to achieve the desired result. Accordingly, polynucleotides of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length are contemplated. Specifically contemplated herein are polynucleotides that are 2 to 30 nucleotides, or 5 to 20 nucleotides, or 6 to 10 nucleotides in length.

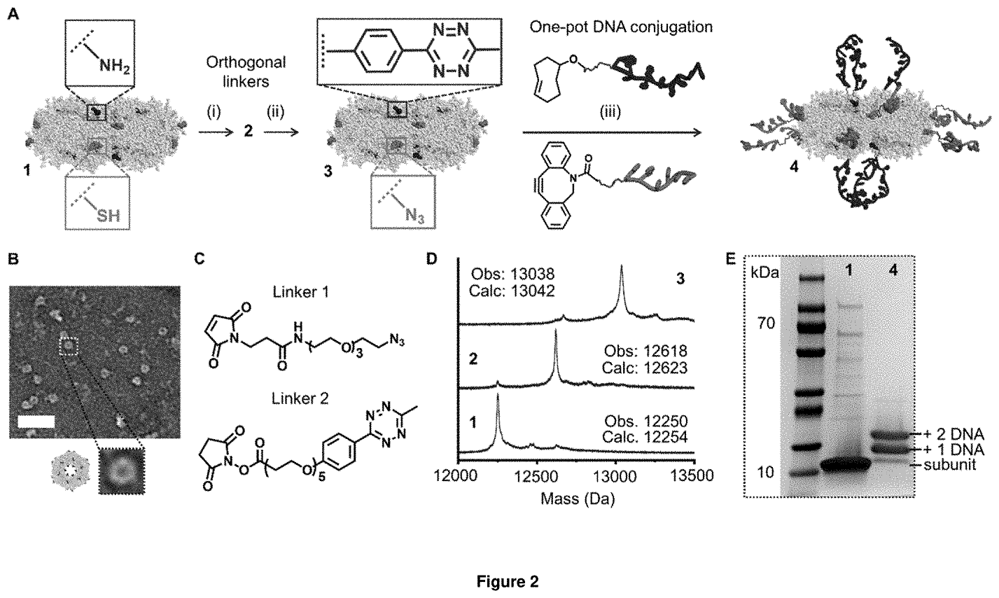

The polynucleotides disclosed herein can be conjugated to a protein disclosed herein. As used herein, the term “conjugated” includes both covalent and non-covalent interactions between the protein and the polynucleotide e.g., covalent conjugation or ligand binding, such as sugar binding (e.g., functionalizing a polynucleotide with a sugar moiety (such as a monosaccharide) such that the polynucleotide-sugar conjugate is attached to a protein via binding of the sugar moiety). Appropriate chemistries for conjugating a polynucleotide to a protein disclosed herein are known to those skilled in the art. Conjugation of the polynucleotide to the protein may be accomplished using, for example, a bio-orthogonal copper catalyzed or copper-free click chemistry reaction or an inverse-electron demand Diels-Alder (IEDDA) reaction. The protein to be conjugated to a polynucleotide may comprise an azide, a tetrazine, or a combination thereof. In some cases, the protein to be conjugated to a polynucleotide comprises one azide. In some cases, the protein to be conjugated to a polynucleotide comprises a plurality of azides. In some cases, the protein to be conjugated to a polynucleotide comprises one tetrazine. In some cases, the protein to be conjugated to a polynucleotide comprises a plurality of tetrazines. In some cases, the protein to be conjugated to a polynucleotide comprises a combination of azides and tetrazines. The azide may be located at the C-terminus or N-terminus of the protein, or it may be an internal azide (e.g., an azide located on the side chain of an amino acid residue in the protein). The azide may be introduced into the protein via an azide-containing linker, e.g., linker 1 or linker 2 of FIG. 2, or via a non-naturally occurring amino acid. The polynucleotide to be conjugated may comprise an alkene or alkyne, which acts as the complimentary click reagent. The alkyne may be introduced into the polynucleotide via a linker containing the alkene or alkyne, e.g., trans-cyclooctene (TCO), dibenzocyclooctyne (DBCO), or bicyclononyne (BCN). Alternatively, the polynucleotide may comprise the azide or tetrazine and the protein may comprise the alkene or alkyne. It is to be understood that other conjugation methods may be used to effect conjugation of the polynucleotide to the protein, e.g., NHS ester conjugation, isocyanate conjugation, isothiocyanate conjugation, maleimide conjugation, iodoacetamide conjugation, and other conjugation methods known to those skilled in the art.

“Hybridization” means an interaction between two strands of nucleic acids by hydrogen bonds in accordance with the rules of Watson-Crick DNA complementarity, Hoogsteen binding, or other sequence-specific binding known in the art. Hybridization can be performed under different stringency conditions known in the art. Under appropriate stringency conditions, hybridization can occur between two polynucleotides that are about 60% or above, about 70% or above, about 80% or above, about 90% or above, about 95% or above, about 96% or above, about 97% or above, about 98% or above, or about 99% or above complementary to each other.

In various aspects, the methods include use of polynucleotides that are 100% complementary to each other, i.e., a perfect match, while in other aspects, the polynucleotides are at least (meaning greater than or equal to) about 95% complementary to each other over the relevant length, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to each other over the relevant length. By relevant length is meant the length of a polynucleotide that hybridizes to another polynucleotide as disclosed herein. For example and without limitation, a polynucleotide strand having 21 nucleotide units can base pair with another polynucleotide of 21 nucleotide units, yet only 19 bases on each strand are complementary or sufficiently complementary, such that the “duplex” has 19 base pairs. The remaining bases may, for example, exist as 5′ and/or 3′ overhangs. Further, within the duplex, 100% complementarity is not required; substantial complementarity is allowable within a duplex. Sufficient complementarity refers, in various embodiments, to 75%, 80%, 85%, 90%, 95%, 99% or 100% complementarity.

### Protein Building Blocks for Hierarchical Structures

A protein was selected as the protein for assembly-stable protein 1 (Sp1, PDB: 1TR0): a symmetric homododecameric protein with pseudo hexagonal-prism geometry. To align the chemical anisotropy of the protein's surface to the shape anisotropy of the protein (FIG. 1A), a mutant (Sp1m) was recombinantly expressed with 24 surface accessible primary amines and 12 thiols located axially and equatorially, respectively (Table 1, below).

TABLE 1

| | |
|---|---|
| Protein | MATRTPKLVKHTLLTRFQDCITREQIDNYINDYTNLLDLIPSMQSFNWGTDLGME |
| sequence | SAELNRGYTHAFESTFESKSGLQEYLDSAALAAFAEGFLPTLSQRLVIDYFLY- |
| ^{a} | (SEQ ID NO: 1) |
| Gene | ATG GCG ACC CGC ACC CCG AAA CTG GTT AAA CAC ACC CTG CTG ACC |
| sequence | CGC TTC CAG GAT TGC ATT ACC CGC GAA CAG ATC GAC AAC TAC ATC |
| | AAC GAC TAC ACC AAC CTG CTG GAT CTG ATT CCG AGC ATG CAG AGC |
| | TTC AAC TGG GGC ACC GAC CTG GGT ATG GAG AGC GCG GAA CTG AAC |
| | CGT GGT TAC ACC CAC GCG TTC GAG AGC ACC TTT GAA AGC AAA AGC |
| | GGC CTG CAG GAG TAT CTG GAT AGC GCG GCG CTG GCG GCG TTT GCG |
| | GAA GGT TTT CTG CCG ACC CTG AGC CAA CGC CTG GTT ATT GAT TAC |
| | TTT CTG TAT TAA (SEQ ID NO: 2) |

^{a} Mutations relative to native protein sequence (1) are highlighted: deletion of native lysines [K18Q and K44Q] (bolded); addition of cysteine [E20C] (underlined).

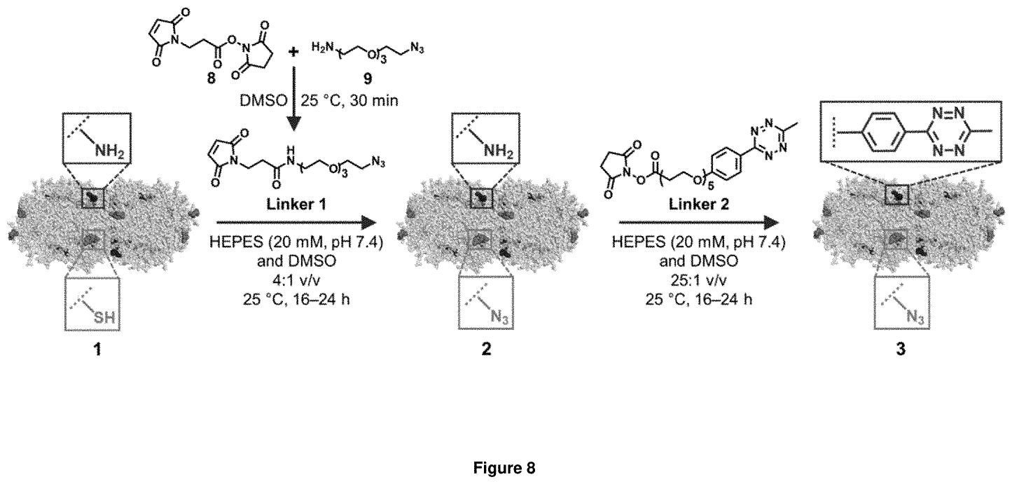

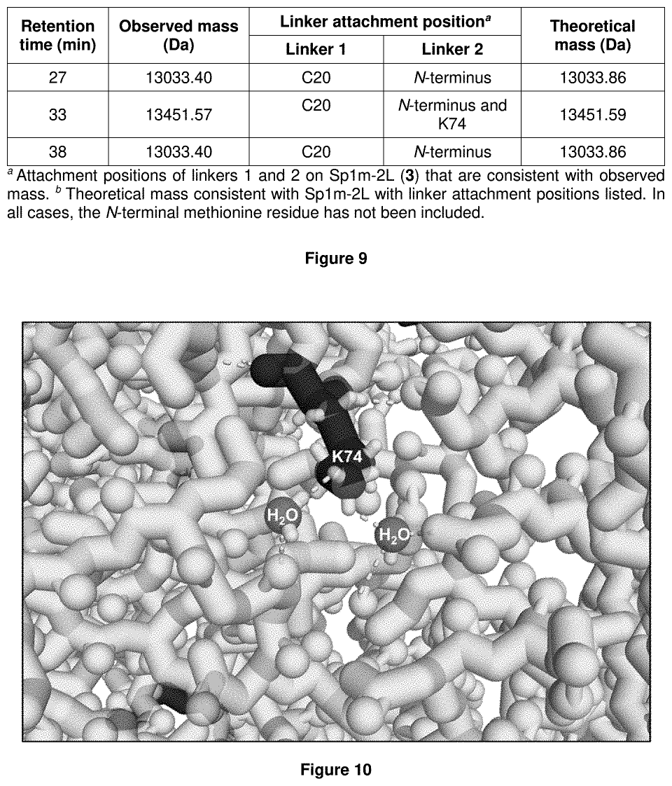

Importantly, this mutant retains the geometry of the native protein as characterized by transmission electron microscopy (TEM, FIG. 2B). The designed chemical anisotropy was then exploited to introduce orthogonal DNA ligands to the axial and equatorial faces (FIG. 2A). In a typical synthesis, the equatorial cysteine residues were first modified with a thiol-reactive hetero-bifunctional crosslinker (Linker 1, FIG. 2C and FIG. 8) to install azide functional groups. Near-complete (>95%) modification of the cysteine residues was confirmed using matrix-assisted laser desorption-ionization time-of-flight mass spectrometry (MALDI-TOF MS, FIG. 2D). The axial primary amines were subsequently reacted with an amine-reactive hetero-bifunctional crosslinker (Linker 2, FIG. 2C) to install tetrazine functional groups. Although there are two primary amines per monomeric subunit (lysine K74 and N-terminus), MALDI-TOF MS analysis indicated high yield (>90%) modification of only a single primary amine per subunit. High resolution, top-down proteomic evaluation of this species revealed that the N-terminal primary amine was modified, with marginal to no functionalization of K74 (FIG. 9). The low reactivity of K74 was attributed to its involvement in hydrogen bonding with an adjacent subunit (FIG. 10).

In some cases, the protein comprises a mutant protein. In some cases, the protein comprises Sp1m. In some cases, the first protein and the second protein are the same, i.e., the first protein and second protein have the same amino acid sequence. In some cases, the first protein and the second protein are different, i.e., the first protein and the second protein have different amino acid sequences.

In some cases, each of the one or more polynucleotides of the patch A is conjugated to an amino acid residue of the first protein. In some cases, each of the one or more polynucleotides of the patch B is conjugated to an amino acid residue of the first protein. In some cases, each of the one or more polynucleotides of the patch A′ is conjugated to an amino acid residue of the second protein. In some cases, each of the one or more polynucleotides of the patch B′ is conjugated to an amino acid residue of the second protein. In some cases, each of the one or more polynucleotides of the patch A and each of the one or more polynucleotides of the patch B is conjugated to an amino acid residue of the first protein. In some cases, each of the one or more polynucleotides of the patch A′ and each of the one or more polynucleotides of the patch B′ is conjugated to an amino acid residue of the second protein. In some cases, the first protein and the second protein each comprise a plurality of amino acid residues conjugated to polynucleotides.

In some cases, the protein has one or more amino acid residues suitable for conjugation to DNA on its surface. In some cases, the amino acid residue is a lysine or a cysteine. In some cases, the amino acid residue is a lysine. In some cases, the amino acid residue is a cysteine. In some cases, the amino acid is an unnatural amino acid residue or other orthogonal amino acid residue, e.g. 4-azido-phenylalanine or 4-(6-methyl-s-tetrazin-3-yl)phenylalanine.

### Generalizable Synthesis of Protein-DNA Conjugates

Having established a synthetic route to prepare Sp1m with two orthogonal functional groups for click chemistry (tetrazines and azides), DNA was then attached to the protein surface. It has been shown that the inverse electron demand Diels-Alder (IEDDA) reaction between tetrazines and trans-cyclooctene (TCO) is sufficiently orthogonal to the copper-free strain-promoted alkyne-azide cycloaddition (SPAAC) reaction between azides and dibenzocyclooctyne (DBCO), such that these reactants may be used simultaneously to achieve selective, multi-target functionalization. Therefore, a one-pot reaction was employed to simultaneously conjugate orthogonal TCO- and DBCO-terminated DNA ligands to the linker-modified protein. Denaturing polyacrylamide gel electrophoresis (PAGE) confirmed successful modification of the protein and revealed the attachment of 1 or 2 DNA ligands per protein subunit (FIG. 2E). To understand this distribution and to confirm the orthogonal reactivity of the two DNA conjugation reactions, the reactions were conducted separately and analyzed via denaturing PAGE. This confirmed that DBCO-DNA ligands react exclusively with the equatorial azides with high conversion (calculated by gel densitometry), resulting in ˜10 DNA ligands in the equatorial plane. The TCO-DNA ligands react with lower conversion, but good selectivity, suggesting that ˜3-4 DNA ligands occupy each axial face of the protein, for a total of 6-8 axial DNA per building block. Without wishing to be bound by theory, the lower conversion may be attributable to the proximity of the N-termini to each other in the inner portion of the structure, which may lead to steric and electrostatic congestion with the bulky, negatively-charged DNA. Given that as few as two closely placed DNA ligands on a protein's surface can act cooperatively to form interface interactions between proteins, a relatively small number of DNA ligands, e.g., 3-4 DNA ligands, per face would be sufficient to define the axial interaction. Overall, this conjugation strategy is highly effective and enables the preparation of 19 unique Sp1m-DNA building blocks.

### Directional Assembly Encoded by Strong Axial- or Equatorial-DNA Interactions

While the above conjugation strategy controls the spatial distribution of DNA ligands on the protein surface, DNA sequence design allows for the specificity and strength of the resulting DNA-DNA interactions to be programmed. DNA sequences that interact orthogonally, in different directions and at distinct stages, can be used to define a multi-step hierarchical assembly pathway driven by the hybridization of complementary DNA (FIG. 1B). To this end, building blocks where the axial and equatorial DNA sequences have disparate melting temperatures (Tₘ) were used, such that directionally specific interactions occur at different temperatures (DNA designs in Table 2, below).

TABLE 2

| | | SEQ | | | | |
|---|---|---|---|---|---|---|
| | | ID | Tₘ | ε₂₆₀ | Calcd MW | Found |
| Name | Sequence (5′ to 3′)^{a} | NO: | (° C.)^{b} | (M⁻¹ cm⁻¹) | (Da) | MW (Da) |
| A_{S} | TCO-CTGGAACTGT | 3 | 44 | 93700 | 3231 | 3227 |
| A_{S}-Cy5 | TCO-Cy5- | 4 | 44 | 103700 | 3760 | 3760 |
| | CTGGAACTGT | | | | | |
| A′_{S} | TCO-ACAGTTCCAG | 5 | 44 | 99300 | 3200 | 3197 |
| A′_{S}-Cy3 | TCO-Cy3- | 6 | 44 | 104230 | 3703 | 3702 |
| | ACAGTTCCAG | | | | | |
| A_{W} | TCO-AATATATT | 7 | 8 | 87100 | 2596 | 2593 |
| A_{W}-Cy5 | TCO-Cy5-AATATATT | 8 | 8 | 97100 | 3129 | 3125 |
| A_{W}-Cy3 | TCO-AATATATT-Cy3 | 9 | 8 | 92030 | 3103 | 3100 |
| A_{NC} | TCO-TTTTTT | 10 | nc | 49200 | 1952 | 1950 |
| A_{NC}- | TCO-Cy5-TTTTTT | 11 | nc | 59200 | 2485 | 2482 |
| Cy5 | | | | | | |
| A_{NC}- | TCO-TTTTTT-Cy3 | 12 | nc | 54130 | 2459 | 2456 |
| Cy3 | | | | | | |
| E_{S} | DBCO-CTACAAATCT | 13 | 35 | 104200 | 3542 | 3536 |
| E_{S}-Cy3 | DBCO-Cy3- | 14 | 35 | 109130 | 4049 | 4042 |
| | CTACAAATCT | | | | | |
| E′_{S} | DBCO-AGATTTGTAG | 15 | 35 | 113400 | 3653 | 3647 |
| E′_{S}-Cy5 | DBCO-Cy5- | 16 | 35 | 123400 | 4186 | 4179 |
| | AGATTTGTAG | | | | | |
| E_{W1} | DBCO-AATATT | 17 | ≤0 | 73000 | 2361 | 2361 |
| E_{W1}- | DBCO-Cy3-AATATT | 18 | ≤0 | 77930 | 2868 | 2865 |
| Cy3 | | | | | | |
| E_{W1}- | DBCO-Cy5-AATATT | 19 | ≤0 | 83000 | 2894 | 2890 |
| Cy5 | | | | | | |
| E_{W2} | DBCO-TAATTA | 20 | ≤0 | 73600 | 2361 | 2362 |
| E_{NC} | DBCO-TTTTTTTT | 21 | nc | 73400 | 2942 | 2940 |
| E_{NC}- | DBCO-Cy3-TTTTTTTT | 22 | nc | 78330 | 3450 | 3443 |
| Cy3 | | | | | | |
| E_{NC}- | DBCO-Cy5-TTTTTTTT | 23 | nc | 83400 | 3476 | 3468 |
| Cy5 | | | | | | |

^{a}Non-standard nucleotides:
TCO (trans-cyclooctene)-2-cyanoethyl (E)-cyclooct-4-enyl N,N-diisopropyl phosphoramidite, synthesized as described in SI Section 2.3. ε₂₆₀ = 0 M⁻¹ cm⁻¹ (i.e., no correction applied).
DBCO (dibenzocyclooctyne)-5′-DBCO-TEG phosphoramidite (Glen Research #10-1941). ε₂₆₀ = 8000 M⁻¹ cm⁻¹.
Cy3-cyanine 3 phosphoramidite (Glen Research #10-5913). ε260 = 4930 M⁻¹ cm⁻¹.
Cy5-cyanine 5 phosphoramidite (Glen Research #10-5915). ε260 = 10000 M⁻¹ cm^{-1.}
^{b}Melting temperatures (Tₘ, rounded to nearest ° C.) were calculated for complementary and self-complementary sequences using the IDT Oligo Analyzer tool, using [DNA] = 1 μM and [Mg²⁺] = 10 mM. Sequences used as non-complementary interactions are indicated by nc.

Specifically, interactions were designed to be either “strong” (Tₘ>>room temperature, RT) or “weak” (Tₘ<<RT). Without wishing to be bound by theory, it is thought that, upon cooling, the strong interactions hybridize first and building blocks undergo a first stage of assembly. This assembled structure display weakly-interacting DNA ligands in a multivalent fashion, resulting in an emergent interaction with enhanced cooperativity and increased Tₘ relative to the isolated weak interactions. The emergent interaction can then drive a second stage of assembly and the formation of a complex assembled structure.

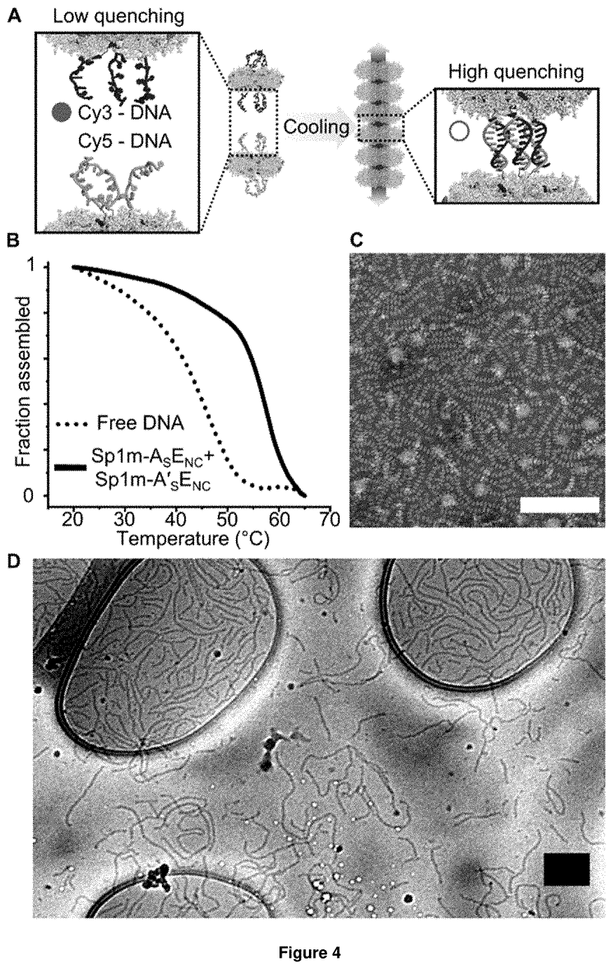

To test if the DNA design strategy imparted directionality on the interactions (axial vs equatorial), the assembly outcomes of systems where only strong interactions are present were initially characterized. Temperature-dependent association of Sp1m-DNA conjugates was probed using a donor-quenching Förster resonance energy transfer (FRET) based technique (FIG. 4A, 4B). In a typical experiment, a pair of complementary Sp1m-DNA conjugates was functionalized with cyanine 3 (Cy3) and cyanine 5 (Cy5) modified DNA, respectively. Without wishing to be bound by theory, as the proteins assemble, the efficiency of FRET from excited Cy3 to Cy5 increases, leading to quenching of Cy3 fluorescence. Therefore, FRET efficiency monitored via the change in Cy3 fluorescence upon cooling from 65 to 20° C., provides a measure of the degree of assembly (Example 6, below). Initially, strong axial interactions (denoted A_{S}) were studied using two complementary conjugates, Sp1m-A_{S}E_{NC} and Sp1m-A′_{S}E_{NC}, with Cy5- and Cy3-modified axial DNA, respectively, and non-complementary equatorial (E_{NC}) interactions that will not assemble equatorially. Their temperature-dependent association profile displayed a single transition with a Tₘ of 57.3° C. and full width half-maximum (FWHM, see Example 6, below) of 10.8° C., compared to Tₘ=43.4° C. and FWHM=16.4° C. for the free DNA duplex (FIG. 4B). The increased Tₘ and decreased FWHM observed for the Sp1m-DNA conjugates, relative to the free DNA duplex, are suggestive of a multivalent and cooperative interaction between proteins.

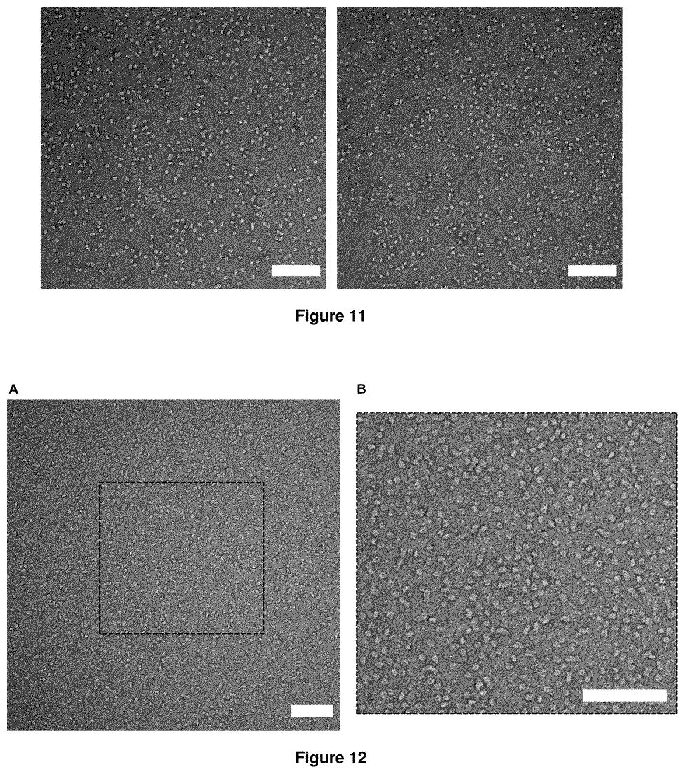

Sp1m-A_{S}E_{NC} and Sp1m-A′_{S}E_{NC} were then slow cooled (0.1° C./10 min) and the assembly products were characterized in the dried and native states using negative stain and cryogenic TEM, respectively (FIG. 4C, 4D). These micrographs revealed the formation of polymeric, 1-dimensional (1 D) protein chains, connected through axial interfaces. Remarkably, in the dried state polymeric structures containing tens of proteins can be resolved (FIG. 4C) and chains measuring several hundred nm long in the native state can be observed (FIG. 4D), with negligible off-target, non-axial interactions. Negative stain TEM of a control sample where only one building block is present (i.e. Sp1m-A_{S}E_{NC}) shows no evidence of assembly (FIG. 11). Taken together, these data support the hypothesis that a strong DNA interaction (defined via sequence design) and the axial functionalization of Sp1m (defined via mutant design and specific functionalization) encodes highly directional interactions between proteins.

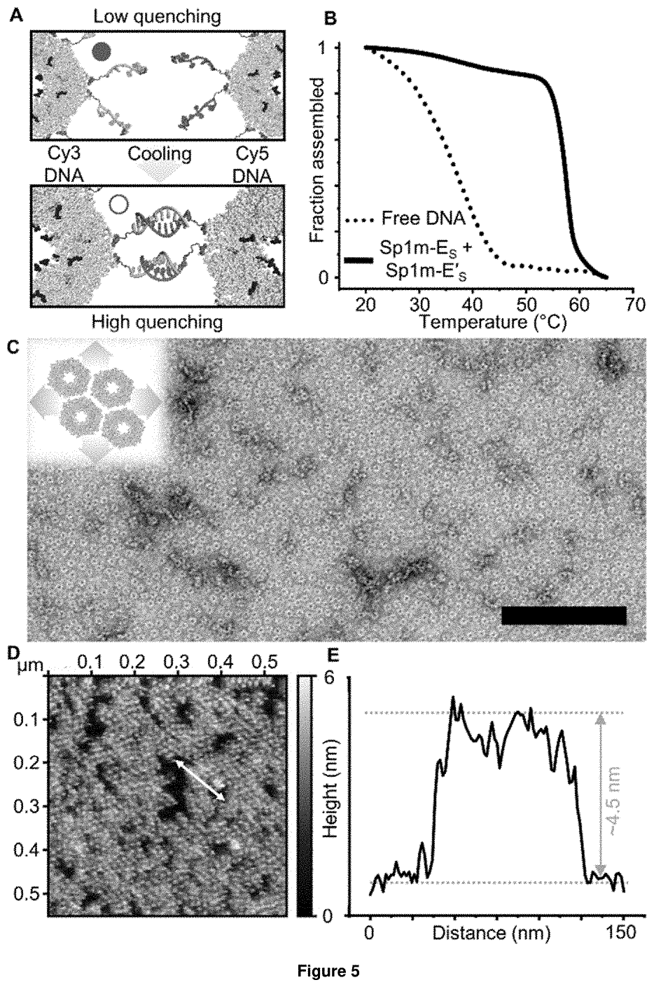

Next, the designed strong equatorial interactions (denoted E_{S}) were interrogated using an identical donor-quenching FRET technique with a pair of complementary Sp1m-DNA conjugates, Sp1m-E_{S} and Sp1m-E′_{S}, functionalized with Cy3- and Cy5-modified DNA, respectively (FIG. 5A). As anticipated, the temperature-dependent association profile for Sp1m-E_{S} and Sp1m-E′_{S} displayed a single, sharp transition (FIG. 5B). Analogous to the strong axial interactions, this transition has a higher Tₘ (57.3° C.) and lower FWHM (4.1° C.) compared to the free DNA duplex (35.9 and 14.0° C., respectively), again suggestive of a multivalent and cooperative interaction between proteins. To assess the directionality of these interactions and characterize the assembly products, Sp1m-E_{S} and Sp1m-E′_{S} were slow cooled (0.1° C./10 min) and observed in the dried state using negative-stain TEM (FIG. 5C) and in their native environment using liquid atomic force microscopy (AFM, FIG. 5D, 5E), which enabled quantification of assembly height. Both techniques revealed 2-dimensional (2D) arrays of assembled proteins, connected through equatorial interfaces, suggesting directional interactions in the equatorial plane. Importantly, negative stain TEM of a control sample comprising only one building block (i.e., Sp1m-E_{S}) shows no evidence of assembly (FIG. 12). Moreover, the formation of monolayer structures was confirmed using AFM (FIG. 5D), which further supports that favorable interactions only exist in the equatorial plane.

In some cases, the polynucleotide moieties comprise a DNA sequence listed in Table 2.

### Multi-Stage Assembly Encoded by Strong and Weak DNA Interactions

Having validated the design for encoding strong, directional interactions between proteins and characterized the assembly behavior resulting from these single-step assembly processes, the investigation next studied systems that could undergo defined, multi-step assembly. Guided by the hypothesis that building blocks with both sufficiently strong and weak surface interactions would be able to traverse a hierarchical assembly pathway that relies on emergent multivalency to induce the second stage of assembly, building blocks were designed displaying axial and equatorial DNA with vastly different interaction strengths, as characterized by Tₘ (Table 2, above). In all cases, the weak interaction comprises self-complementary DNA sequences with a theoretical Tₘ<10° C., to ensure negligible association at ambient temperature prior to undergoing the first stage of assembly. To characterize these assembly steps, a donor-quenching FRET based technique was again used to capture their assembly profiles as a function of temperature.

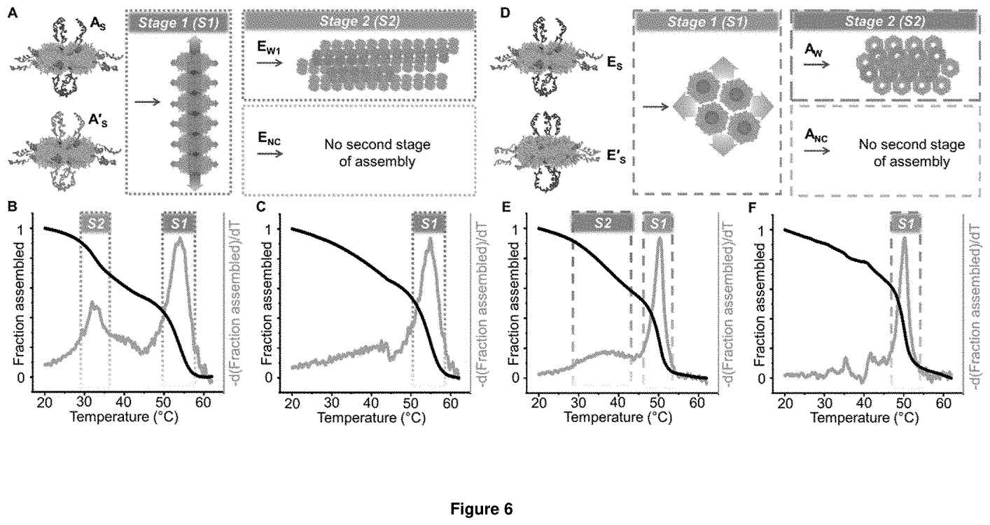

A pair of Sp1m building blocks, Sp1m-A_{S}E_{W1} and Sp1 m-A′_{S}E_{W1}, were synthesized in which the proteins were functionalized at the axial positions with the previously discussed strong DNA sequences (A_{S} and A′_{S}) and at the equatorial positions with a self-complementary weak DNA sequence (E_{W1}). The equatorial DNA sequences of Sp1 m-A_{S}E_{W1} and Sp1m-A′_{S}E_{W1} were modified with Cy3 and Cy5 dyes, respectively, such that upon the formation of 1D protein chains, driven by the strong axial interactions, the proximity of equatorial DNA increases and thus partial quenching of the Cy3 fluorescence occurs. Without wishing to be bound by theory, further quenching takes place when the 1D structures associate through hybridization of equatorial DNA stands, indicating a second stage of assembly. As a control, an additional pair of building blocks, Sp1m-A_{S}E_{NC} and Sp1m-A′_{S}E_{NC}, was synthesized whereby the equatorial DNA ligands of Sp1m-A_{S}E_{NC} and Sp1m-A′_{S}E_{NC} were modified with Cy3 and Cy5 dyes, respectively. The degree of assembly for both systems was determined by measuring the fluorescence of Cy3 upon cooling from 65 to 20° C. (FIG. 6A, 6B). The assembly profiles of both sets of building blocks revealed a sharp transition at Tₘ=54° C., consistent with the Tₘ measured for the assembly of axial-only system (57.3° C.), that can be attributed to the association of proteins through axial interactions. The discrepancy in Tₘ is due to the difference in salt concentration between experiments. Additionally, for the building blocks modified with self-complementary equatorial DNA (Sp1m-A_{S}E_{W1} and Sp1 m-A′_{S}E_{W1}) a second transition occurs. This transition has a Tₘ of 32.7° C., which is greater than expected for the free six base-pair (bp) E_{W1} duplex (theoretical Tₘ<5° C.), indicating a highly cooperative assembly event.

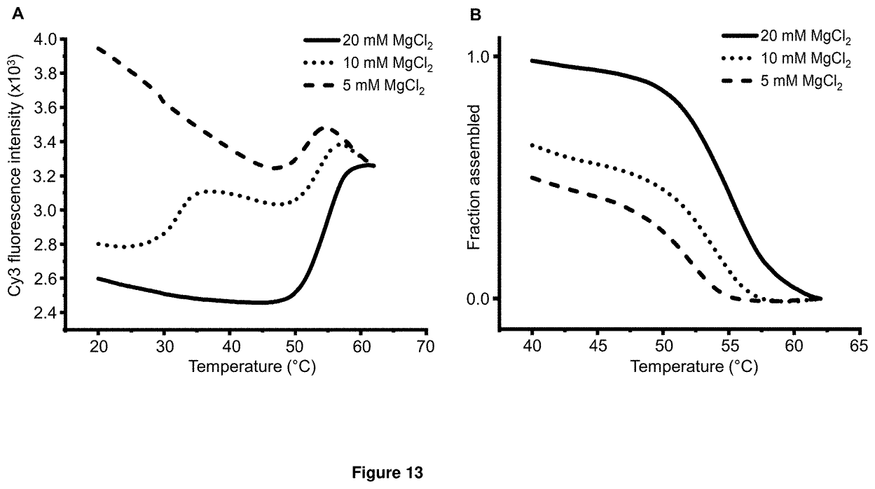

DNA interactions are greatly influenced by their ionic environment, and thus the influence of different salt conditions in this two-step assembly profile was studied. The cooling experiment was repeated at a higher and lower salt concentration (20 mM and 5 mM vs 10 mM MgCl₂, FIG. 13). Interestingly, in both 5 and 20 mM MgCl₂, the transition at 32.7° C. disappeared and the assembly profiles displayed a single transition at 52.0 and 55.2° C., respectively, but these conditions resulted in significantly different relative fractions assembled (FIG. 13B). Assembly driven by axial interactions resulted in a much greater fraction assembled in 20 mM MgCl₂ compared to lower salt concentrations, suggesting that at high salt concentration, the two assembly steps become concerted and cannot be resolved. At the lowest salt concentration (5 mM), the assembly profile suggests that only the first (axial) stage of assembly occurred and that a salt concentration between 5 and 20 mM is required for both assembly stages to occur and be resolvable. These trends are consistent with the influence of ionic environment on the hybridization of DNA; however, it is notable that the two stages of assembly differ substantially in the extent to which they are influenced by changes in salt concentration, therefore pointing to additional methods to fine tune hierarchical assembly pathways. Overall, this set of experiments provides evidence for a temperature-dependent, programmed, multi-step assembly pathway defined by DNA interactions and supports the hypothesis that Sp1m-DNA conjugates assemble first through axial interactions and then through equatorial interactions. Importantly, this second stage of assembly relies on an emergent interaction that is encoded by DNA sequences in the initial building block but is only activated after the first assembly step. This process is akin to the hierarchical generation of tertiary and quaternary protein structures defined exclusively by the information present in the primary amino acid sequence.

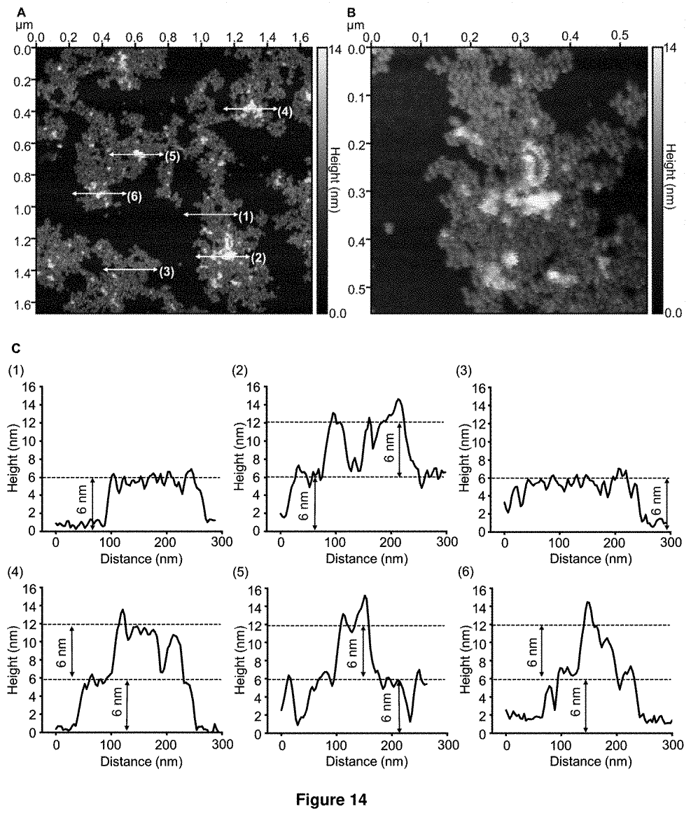

Next, an investigation was undertaken to study whether a reversed assembly pathway could be programmed by simply switching the relative strengths of DNA interactions at the axial and equatorial positions. Accordingly, a new set of building blocks, Sp1m-A_{W}E_{S} and Sp1m-A_{W}E′_{S}, was synthesized employing the previously discussed strong equatorial complementary DNA sequences (E_{S} and E′_{S}) as well as weak self-complementary DNA sequences at the axial positions (A_{W}). The axial DNA sequences of Sp1m-A_{W}E_{S} and Sp1m-A_{W}E′_{S} were modified with Cy3 and Cy5 dyes, respectively, where partial quenching for the first stage of assembly was expected (formation of 2D structures through strong equatorial interactions), and further quenching upon subsequent axial interactions during cooling from 65 to 20° C. To provide a comparison where axial interactions are inhibited, Sp1m-A_{NC}E_{S} and Sp1m-A_{NC}E′_{S} were synthesized with non-complementary axial DNA ligands (A_{NC}) modified with Cy3 and Cy5 dyes, respectively. When comparing the temperature-dependent assembly profiles for these two sets of building blocks, the system containing both interaction types (Sp1m-A_{W}E_{S} and Sp1m-A_{W}E′_{S}) displayed two distinct transitions (Tₘ=50.4 and 38.1° C.) whereas the system with A_{NC} interactions displayed only a single transition (50.4° C.; FIG. 6C, 6D). Without wishing to be bound by theory, the common transition at 50.4° C. can be attributed to the initial association of proteins in the equatorial plane to form 2D structures and the unique transition at 38.1° C. to the subsequent onset of axial interactions between these 2D structures. The transition at 38.1° C. is relatively broad, compared to the first assembly step, which may be due to the polydispersity of structures that associate in this step (FIG. 14). Together, these experiments support the hypothesis that Sp1m-A_{W}E_{S} and Sp1m-A_{W}E′_{S} undergo a reversed, thermally controlled, multi-step assembly pathway, first associating through equatorial interactions and then via axial interactions.

In some cases, the polynucleotides of the hierarchical protein structures disclosed herein are contained in at least two patches on each protein. As used herein the term “patch” refers to a grouping of one or more polynucleotides that are conjugated to the surface of a protein, and which are capable of interacting (e.g., hybridizing) with one or more groupings of one or more polynucleotides that are conjugated to the surface of one or more other proteins. In some cases, one or more polynucleotides that are conjugated to the surface of a protein are capable of interacting with one or more groupings of one or more polynucleotides that are conjugated to the surface of one other protein. In some cases, a grouping of one or more polynucleotides that are conjugated to the surface of a protein are capable of interacting with one or more groupings of one or more polynucleotides that are conjugated to the surface of more than one other protein. In some cases, a grouping of one or more polynucleotides that are conjugated to the surface of a first protein are capable of interacting with one or more groupings of one or more polynucleotides that are conjugated to the surface of a second protein. In some cases, one or more polynucleotides contained in a patch along the axial plane of a protein is capable of hybridizing to one or more polynucleotides contained in a patch along the axial plane of another protein. In some cases, one or more polynucleotides contained in a patch along the equatorial plane of a protein is capable of hybridizing to one or more polynucleotides contained in a patch along the equatorial plane of another protein. In some cases, one or more polynucleotides contained in a patch along the axial plane of a protein is capable of hybridizing to one or more polynucleotides contained in a patch along the equatorial plane of another protein. Thus, the interactions between the one or more polynucleotides contained in a patch on a protein and the one or more polynucleotides contained in a patch on another protein can be spatially defined, thereby creating the hierarchical protein structure. By way of non-limiting example, one can design nucleic acid sequences such that polynucleotides contained in patches in axial planes of two proteins hybridize at a different melting temperature relative to polynucleotides contained in patches in equatorial planes of the two proteins, such that modulation of the temperature during assembly directs the sequential assembly of a hierarchical protein structure along a specific multi-step pathway. In some cases, the first protein has a first and a second plane, the second protein has a first and a second plane, and the first plane of the first protein and the first plane of the second protein and the second plane of the first protein and the second plane of the second protein comprise different amino acid residues that allow for orthogonal conjugation of different polynucleotides along the first plane of the first protein and the first plane of the second protein relative to polynucleotides along the second plane of the first protein and the second plane of the second protein.

In some cases, each patch comprises about 1 to about 1000, about 1 to about 500, about 1 to about 100, about 1 to about 50, about 1 to about 20, about 1 to about 10, or about 1 to about 5 polynucleotides. In some cases, a plurality of polynucleotides is contained within a patch. In some cases, each of the plurality of polynucleotides contained within at least one of the one or more patches has the same nucleic acid sequence. In some cases, at least two polynucleotides contained within at least one of the one or more patches have different nucleic acid sequences. In some cases, the plurality of polynucleotides contained within a first patch has a melting temperature different to a melting temperature of the plurality of polynucleotides of a second patch. In some cases, a patch consists of one polynucleotide. In some cases, the one polynucleotide in a first patch has a melting temperature different to a melting temperature of a polynucleotide of a second patch. In some cases, the polynucleotides comprise a sequence listed in Table 2.

In some aspects, a method of making a hierarchical protein structure of the disclosure is provided, comprising contacting: (a) a first protein comprising: (i) a patch A comprising one or more polynucleotides conjugated to the surface of the first protein; and (ii) a patch B comprising one or more polynucleotides conjugated to the surface of the first protein; and (b) a second protein comprising: (i) a patch A′ comprising one or more polynucleotides conjugated to the surface of the second protein; and (ii) a patch B′ comprising one or more polynucleotides conjugated to the surface of the second protein; wherein the one or more polynucleotides of the patch A is sufficiently complementary to the one or more polynucleotides of the patch A′ to hybridize, and wherein the contacting is performed under conditions that result in the one or more polynucleotides of the patch A hybridizing to the one or more polynucleotides of the patch A′, thereby making the hierarchical protein structure.

In some cases, the patch A is conjugated to the surface of the first protein along a first plane in space; and the patch B is conjugated to the surface of the first protein along a second plane in space. In some cases, the patch A′ is conjugated to the surface of the second protein along a first plane in space; and the patch B′ is conjugated to the surface of the second protein along a second plane in space. In some cases, the one or more polynucleotides of the patch A are in about the same plane as the one or more polynucleotides of the patch B. In some cases, the one or more polynucleotides of the patch A are in a different plane as the one or more polynucleotides of the patch B. In some cases, the one or more polynucleotides of patch A are orthogonal to the one or more polynucleotides of the patch B. In some cases, the one or more polynucleotides of the patch A′ are in about the same plane as the one or more polynucleotides of the patch B′. In some cases, the one or more polynucleotides of the patch A′ are in a different plane as the one or more polynucleotides of the patch B′. In some cases, the one or more polynucleotides of the patch A′ are orthogonal to the one or more polynucleotides of the patch B′. In some cases, the one or more polynucleotides of the patch A and the one or more polynucleotides of the patch A′ are complementary to each other, and are orthogonal to the one or more polynucleotides of the patch B and the one or more polynucleotides of the patch B′.

In some cases, the one or more polynucleotides of the patch A and the one or more polynucleotides of the patch B comprises DNA, RNA, or a combination thereof. In some cases, the one or more polynucleotides of the patch A′ and each of the one or more polynucleotides of the patch B′ comprises DNA, RNA, or a combination thereof. In some cases, the one or more polynucleotides of the patch A have a different melting temperature than the one or more polynucleotides of the patch B. In some cases, the one or more polynucleotides of the patch A have a higher melting temperature than the one or more polynucleotides of the patch B. the one or more polynucleotides of the patch A′ have a different melting temperature than the one or more polynucleotides of the patch B′. In some cases, the one or more polynucleotides of the patch A′ have a higher melting temperature than the one or more polynucleotides of the patch B′. In some cases, the one or more polynucleotides of the patch A comprise DNA. In some cases, the one or more polynucleotides of the patch A′ comprise DNA. In some cases, the one or more polynucleotides of the patch B comprise DNA. In some cases, the one or more polynucleotides of the patch B′ comprise DNA.

In some cases, the patch A comprises a plurality of polynucleotides and each of the plurality of polynucleotides has the same nucleic acid sequence. In some cases, the patch A comprises a plurality of polynucleotides and at least two polynucleotides contained within the plurality of polynucleotides have different nucleic acid sequences. In some cases, the patch B comprises a plurality of polynucleotides and each of the plurality of polynucleotides has the same nucleic acid sequence. In some cases, the patch B comprises a plurality of polynucleotides and at least two polynucleotides contained within the plurality of polynucleotides have different nucleic acid sequences. In some cases, the patch A′ comprises a plurality of polynucleotides and each of the plurality of polynucleotides has the same nucleic acid sequence. In some cases, the patch A′ comprises a plurality of polynucleotides and at least two polynucleotides contained within the plurality of polynucleotides have different nucleic acid sequences. In some cases, the patch B′ comprises a plurality of polynucleotides and each of the plurality of polynucleotides has the same nucleic acid sequence. In some cases, the patch B′ comprises a plurality of polynucleotides and at least two polynucleotides contained within the plurality of polynucleotides have different nucleic acid sequences.

In some cases, the one or more polynucleotides of the patch A are complementary to the one or more polynucleotides of the patch A′. In some cases, the one or more polynucleotides of the patch A are complementary to the one or more polynucleotides of the patch A of another protein. In some cases, the one or more polynucleotides of the patch B are complementary to the one or more polynucleotides of the patch B′. In some cases, the one or more polynucleotides of the patch B are complementary to the one or more polynucleotides of the patch B of another protein.

In some cases, the hierarchical protein structures further comprise a third protein comprising a patch B′ comprising one or more polynucleotides conjugated to the surface of the third protein which hybridizes to the patch B of the first protein or the patch B′ of the second protein.

In some aspects, the disclosure provides methods of making a hierarchical protein structure of the disclosure is provided, comprising contacting: (a) a first protein comprising: (i) a patch A comprising one or more polynucleotides conjugated to the surface of the first protein; and (ii) a patch B comprising one or more polynucleotides conjugated to the surface of the first protein; and (b) a second protein comprising: (i) a patch A′ comprising one or more polynucleotides conjugated to the surface of the second protein; and (ii) a patch B′ comprising one or more polynucleotides conjugated to the surface of the second protein; wherein the one or more polynucleotides of the patch A is sufficiently complementary to the one or more polynucleotides of the patch A′ to hybridize, and wherein the contacting is performed under conditions that result in the one or more polynucleotides of the patch A hybridizing to the one or more polynucleotides of the patch A′, thereby making the hierarchical protein structure.

In some cases, the one or more polynucleotides of the patch B is sufficiently complementary to the one or more polynucleotides of the patch B′ to hybridize under said conditions. In some cases, the one or more polynucleotides of the patch A and the one or more polynucleotides of the patch A′ have a melting temperature different to the melting temperature of the one or more polynucleotides of the patch B and the one or more polynucleotides of the patch B′. In some cases, the methods further comprise hybridizing the one or more polynucleotides of the patch B to the one or more polynucleotides of the patch B′. In some cases, hybridization of the one or more polynucleotides of the patch A to the one or more polynucleotides of the patch A′ occurs at a different temperature than hybridization of the one or more polynucleotides of the patch B to the one or more polynucleotides of the patch B′. In some cases, wherein hybridization of the one or more polynucleotides of the patch A to the one or more polynucleotides of the patch A′ occurs at a higher temperature than hybridization of the one or more polynucleotides of the patch B to the one or more polynucleotides of the patch B′. In some cases,

In some cases, the one or more polynucleotides of the patch A hybridizes to the one or more polynucleotides of the patch A′ before the one or more polynucleotides of the patch B hybridizes to the one or more polynucleotides of the patch B′. In some cases, hybridization of the one or more polynucleotides of the patch A to the one or more polynucleotides of the patch A′ enables hybridization of the one or more polynucleotides of the patch B to the one or more polynucleotides of the patch B′.

In some cases, the methods further comprise contacting a third protein comprising a patch B′ comprising one or more polynucleotides conjugated to the surface of the third protein wherein the one or more polynucleotides of the patch B′ are sufficiently complementary to the one or more polynucleotides of the patch B of the first protein or the patch B′ of the second protein to hybridize, and wherein the contacting is performed under conditions that result in the one or more polynucleotides of the patch B′ of the third protein hybridizing to the one or more polynucleotides of the patch B of the first protein or the patch B′ of the second protein, thereby making the hierarchical protein structure.

In some cases, assembly of the hierarchical protein structure can occur in one or more directions. In some cases, assembly of the hierarchical protein structure can occur in one direction. In some cases, assembly of the hierarchical protein structure can occur in more than one direction. In some cases, assembly of the hierarchical protein structure can occur in a first direction. In some cases, assembly of the hierarchical protein structure can occur in a second direction. In some cases, assembly of the hierarchical protein structure can occur in a first direction and then in a second direction. In some cases, In some cases, assembly of the hierarchical protein structure can occur in a first direction and then in a second direction upon a temperature change.

### Programming Structural Outcomes Via DNA Design

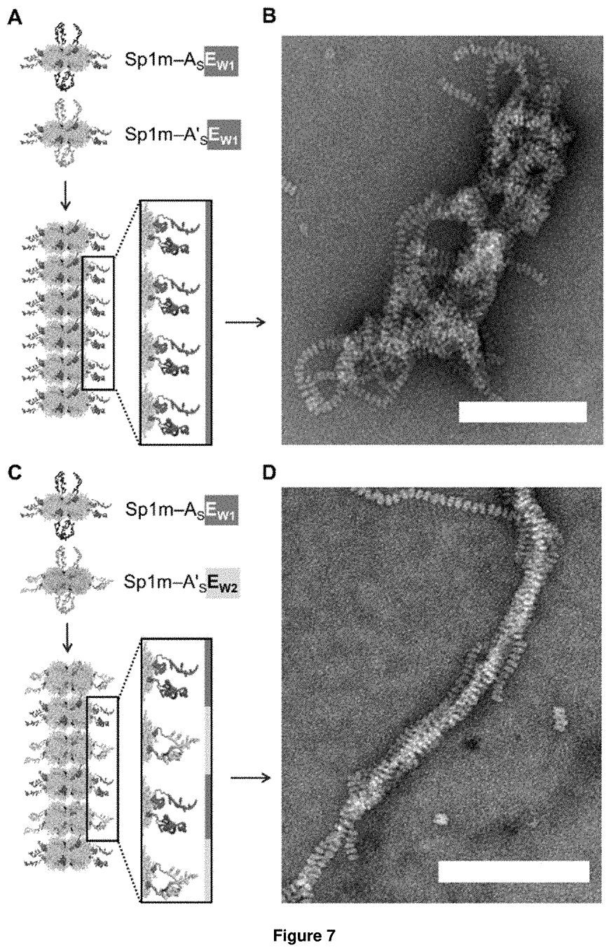

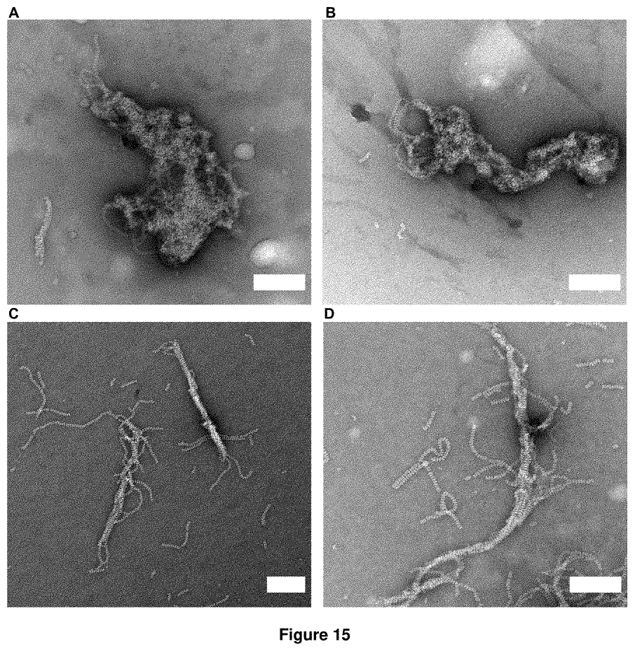

Designing the relative strength of DNA ligands and their spatial arrangement on the protein surface directs assembly along different pathways with distinct assembly outcomes. It was next explored whether the assembly outcome could be changed while maintaining the same pathway, via DNA sequence design. To that end, the structures that arise from an axial-first, equatorial-second assembly pathway were characterized. In addition to the previously described system, Sp1m-A_{S}E_{W1} and Sp1m-A′_{S}E_{W1} (FIG. 4), an additional building block was designed, Sp1m-A′_{S}E_{W2}, where the equatorial sites of the second building block were modified with a weak self-complementary sequence (E_{W2}) orthogonal to E_{W1}. The E_{W1} and E_{W2} DNA sequences (Table 2, above) are identical in length and base pair composition to ensure that differences in the assembly outcome result from differences in the presentation of the emergent second interaction, rather than inherent differences in the interaction strength between the two self-complementary DNA designs. The building blocks were slow cooled (0.1° C./10 min) in two combinations: Sp1m-A_{S}E_{W1} with Sp1m-A′_{S}E_{W1} (FIG. 7A), and Sp1m-A_{S}E_{W1} with Sp1m-A′_{S}E_{W2} (FIG. 7C). The complementarity of A_{S} and A′_{S} ensures that, in the latter system, E_{W1} and Ewe are presented alternately (FIG. 7C). TEM characterization of both samples reveals the formation of 1D protein chains, formed via A_{S} interactions, that interact with each other, suggesting that these two systems traverse the same assembly pathway. However, the two sets of building blocks gave significantly different structural outcomes (FIG. 7B, 7D). For the system containing only E_{W1}-based building blocks, the 1D protein chains had a high propensity to form bundles and fold up upon themselves via intra-chain interactions (FIG. 7A). However, when one of the building blocks is modified with Ewe, the 1D protein chains instead interacted to form elongated filaments. Moreover, TEM suggested that registry between the proteins in each chain was better enforced in this sample (FIG. 15). This highlights how two, orthogonal, self-complementary E_{W} sequences decrease the propensity for the 1D protein chains to fold and bundle and is a key demonstration of how DNA design can not only define a specific assembly pathway but also direct the final structural outcome.

### EXAMPLES

### Example 1: Functionalization of Sp1m with Azide and Tetrazide Linkers

Maleimide-azide linker (Linker 1) was prepared from azido-PEG3-amine (2 μL) in DMSO (48 μL) and 3-maleimido-propionic NHS ester (2.5 mg) in DMSO (50 μL). The mixture was shaken at 650 rpm at 25° C. for 30 min. The reaction was quenched by addition of Tris (1 M, pH 7, 10 μL) and shaken for a further 5 min. The mixture (110 μL) was added to an aliquot of Sp1m (1, 400 μL, 5 μM) and shaken overnight at 650 rpm at 25° C. The reaction mixture was purified by size exclusion chromatography and fractions containing Sp1m-N₃ (2) were pooled, concentrated to 5 μM, and portioned into 1.5 mL Eppendorf Tubes® in 500 μL aliquots. To each aliquot, a solution of methyltetrazine-PEGS-NHS ester (Linker 2, 0.6 μL) in DMSO (20 μL) was added and thoroughly mixed by pipette aspiration. The solution was shaken at 650 rpm for 20 h at 25° C. The reaction mixture was purified by size exclusion chromatography and fractions containing protein were pooled. Sp1m with both azide and tetrazine linkers (Sp1m-2L, 3) was typically reacted with DNA immediately, although Sp1m-2L (3) could be stored at 4° C. for 24 h without loss in reactivity.

### Example 2: DNA Conjugation to Sp1m-2L (3)

DNA conjugation reactions were typically performed on the 0.5, 0.7, or 1 nmol scale with respect to Sp1m-2L (3). A mixture of Sp1m-2L (1 equiv), TCO-DNA (180 equiv), and DBCO-DNA (150 equiv) in HEPES (20 mM, pH 7.4) and NaCl (500 mM) was shaken at 650 rpm for 20 h at 37° C. Unreacted DNA was removed by washing the reaction mixture three times in a 4 mL centrifugal filter with 20 mM HEPES (30 K MWCO, 3000×g, 4° C., 3 min cycles). The reaction mixture was purified by size exclusion chromatography and fractions containing protein were pooled and stored at 4° C.

### Example 3: Donor-Quenching FRET Studies

Combinations of Sp1m-DNA conjugates at 300 nM total Cy3 concentration were mixed (1:1 ratio, 50 μL) and placed in a 96-well plate, heated at 65° C. for 5 min, and then cooled from 65° C. to 20° C. at 0.1° C./0.5 min using a Bio-Rad CFX96 Touch™ real time PCR system. All samples were measured in triplicate, and the data reported represents the average of the three runs. Cy3 fluorescence was measured at 0.1° C. intervals.

Plots of fraction assembled vs temperature were obtained by measuring the fluorescence intensity (I) of two samples: a sample where the donor fluorophore (Cy3) is in the presence of a FRET acceptor (Cy5) (I_{DA}) and a sample where only the donor fluorophore (Cy3) is present (I_{D}). Comparing the fluorescence of both systems allows for the assembly-dependent FRET quenching of the donor to be distinguished from the inherent temperature-dependent change in fluorescence of the donor. From the raw intensity profiles, the temperature-dependent FRET efficiency was determined as:

FRET efficiency=1−I_{DA}/I_{D}

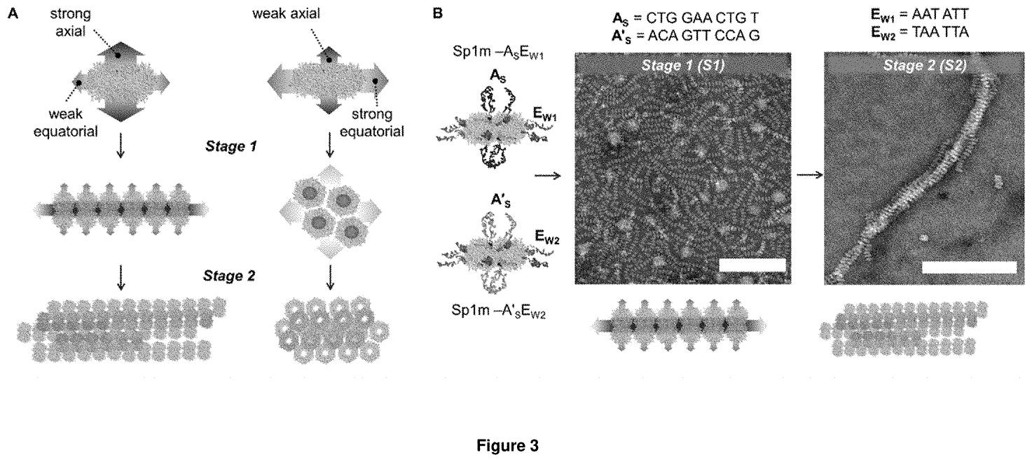

Using the FRET efficiency, “fraction assembled” was defined by taking the maximum FRET ratio as fraction assembled=1 and the minimum FRET ratio as fraction assembled=0 (6). This method was used to generate all plots in FIGS. 2 to 4. Since each system has different assembly outcomes/end-points, the fraction assembled is defined independently for each system.

The data from fraction assembled vs temperature plots were fit with a sigmoidal curve using the “Sigmoidal Fit” function in Origin Pro® from which the 1^{st} derivative was calculated (Fig. S11, solid lines). The derivatized data were subsequently fit with a gaussian curve using the “Single Peak Fit” function in OriginPro®. Melting temperatures (Tₘ) were taken as the peak of the fitted gaussian and the full-width half-maximum (FWHM) was also measured.

### Example 4: Assembly of Sp1m-DNA Conjugates Via Slow-Cooling

Samples were mixed to a total protein concentration of 100 or 500 nM and then cooled from 60° C. to 21° C. at a rate of 0.1° C./10 min using a ProFIex™ PCR system (Applied Biosystems). The resulting structures were characterized using negative-stain TEM, cryo-TEM or AFM.

To obtain negative-stain TEM images, 4 μL of slow-cooled sample (diluted to 100 nM if necessary) were adsorbed onto a glow-discharged carbon-coated Cu grid (Ted Pella) for 2 min. Excess liquid was wicked away by applying filter paper to the underside of the grid. A solution (4 μL) of either 2% uranyl acetate or 0.75% uranyl formate stain (Electron Microscopy Solutions) was applied for 1 min. The sample was allowed to air dry for 10 min after wicking away excess stain. Images were collected on a JEOL 1230 transmission electron microscope at 100 or 120 kV accelerating voltage.

Cryogenic TEM images were obtained by depositing 4 μL of 500 nM sample on a glow-discharged lacey carbon-coated grid (Ted Pella) and plunge-frozen using a FEI Vitrobot Mark IV™ using a blot time of 5 s at 10° C. and high humidity. Images were collected on a Hitachi HT-7700™ Biological S/TEM at 100 kV accelerating voltage.

To obtain AFM images, 5 μL of 500 nM sample were deposited on a freshly cleaved mica substrate. 10 μL of buffer (10 mM MgCl₂, 20 mM HEPES pH 7.4) was added to the substrate and the sample was left to incubate overnight in a high humidity environment to minimize evaporation. All AFM images were captured in ScanAsyst™ PeakForce Tapping™ mode on a BioScope Resolve™ AFM (Bruker) using a SCANASYST-FLUID+™ probe. The effective imaging force ranged from 100 to 200 pN, within the typical force range for AFM imaging of biomolecules.

### REFERENCES

1. C. Gotti et al., Appl. Mater. Today 20, 100772 (2020).
2. A. W. P. Fitzpatrick et al., Proc. Natl. Acad. Sci. U.S.A. 110, 5468-5473 (2013).
3. M. Gale et al., Biophys. J. 68, 2124-2128 (1995).
4. Y. Okada et al., Mol. Gen. Genet. 114, 205-213 (1972).
5. P. Fratzl et al., Prog. Mater. Sci. 52, 1263-1334 (2007).
6. C. A. Mirkin et al., Nature 382, 607-609 (1996).
7. R. J. Macfarlane et al., Science 334, 204-208 (2011).
8. C. R. Laramy et al., Nat. Rev. Mater. 4, 201-224 (2019).
9. D. Morphew et al., ACS Nano 12, 2355-2364 (2018).
10. T. Schnitzer et al., ACS Cent. Sci. 6, 2060-2070 (2020).
11. T. K. Haxton et al., Soft Matter 9, 6851 (2013).
12. A. B. Rao et al., ACS Nano 14, 5348-5359 (2020).
13. H. Sun et al., Front. Bioeng. Biotechnol. 8, 295 (2020).
14. J. B. Bale et al., Science 353, 389-394 (2016).
15. H. Shen et al., De novo design of self-assembling helical protein filaments. Science 362, 705-709 (2018).
16. P. A. Sontz et al., J. Am. Chem. Soc. 137, 11598-11601 (2015).
17. H. Garcia-Seisdedos et al., Nature 548, 244-247 (2017).
18. L. A. Churchfield et al., Acc. Chem. Res. 52, 345-355 (2019).
19. C. Si et al., Chem. Commun. 52, 2924-2927 (2016).
20. J. A. Modica et al., J. Am. Chem. Soc. 140, 6391-6399 (2018).
21. S. Burazerovic et al., Angew. Chem., Int. Ed. 46, 5510-5514 (2007).
22. I. Insua et al., J. Am. Chem. Soc. 142, 300-307 (2020).
23. Z. Zhao et al., Nat. Commun. 12, 589 (2021).
24. J. R. McMillan et al., Acc. Chem. Res. 52, 1939-1948 (2019).
25. J. D. Brodin et al., Proc. Natl. Acad. Sci. 112, 4564-4569 (2015).
26. J. R. McMillan et al., J. Am. Chem. Soc. 139, 1754-1757 (2017).
27. D. Kashiwagi et al., J. Am. Chem. Soc. 140, 26-29 (2018).
28. J. R. McMillan et al., J. Am. Chem. Soc. 140, 6776-6779 (2018).
29. P. H. Winegar et al., Chem 6, 1007-1017 (2020).
30. D. Kashiwagi et al., J. Am. Chem. Soc. 142, 13310-13315 (2020).
31. O. G. Hayes et al., J. Am. Chem. Soc. 140, 9269-9274 (2018).
32. J. R. Mcmillan et al., J. Am. Chem. Soc. 140, 15950-15956 (2018).
33. O. Dgany et al., J. Biol. Chem. 279, 51516-51523 (2004).
34. M. L. Blackman et al., J. Am. Chem. Soc. 130, 13518-13519 (2008).
35. B. L. Oliveira et al., Chem. Soc. Rev. 46, 4895-4950 (2017).
36. N. J. Agard et al., J. Am. Chem. Soc. 126, 15046-15047 (2004).
37. M. R. Karver et al., Angew. Chem., Int. Ed. 51, 920-922 (2012).
38. B. Sacca et al., Nat. Protoc. 4, 271-285 (2009).
39. D. Samanta et al., J. Am. Chem. Soc. 141, 19973-19977 (2019).

1. A hierarchical protein structure comprising two or more proteins extending in one or more dimensions, the hierarchical protein structure comprising:
a first protein comprising: (i) a patch A comprising one or more polynucleotides conjugated to the surface of the first protein; and (ii) a patch B comprising one or more polynucleotides conjugated to the surface of the first protein; and
a second protein comprising: (i) a patch A′ comprising one or more polynucleotides conjugated to the surface of the second protein; and (ii) a patch B′ comprising one or more polynucleotides conjugated to the surface of the second protein;
wherein the one or more polynucleotides of the patch A hybridizes to the one or more polynucleotides of the patch A′, and/or the one or more polynucleotides of the patch B hybridizes to the one or more polynucleotides of the patch B′ to form the hierarchical protein structure.

2. The hierarchical protein structure of claim 1, wherein the one or more polynucleotides of the patch A hybridizes to the one or more polynucleotides of the patch A′, and the one or more polynucleotides of the patch B hybridizes to the one or more polynucleotides of the patch B′ to form the hierarchical protein structure.

3. The hierarchical protein structure of claim 1, wherein each of the one or more polynucleotides of the patch A and each of the one or more polynucleotides of the patch B is conjugated to an amino acid residue of the first protein, and/or wherein each of the one or more polynucleotides of the patch A′ and each of the one or more polynucleotides of the patch B′ is conjugated to an amino acid residue of the second protein.

4. (canceled)

5. The hierarchical protein structure of claim 3, wherein the amino acid residue is a lysine or a cysteine.

6. The hierarchical protein structure of claim 1, wherein the patch A is conjugated to the surface of the first protein along a first plane in space; and the patch B is conjugated to the surface of the first protein along a second plane in space, and/or wherein the patch A′ is conjugated to the surface of the second protein along a first plane in space; and the patch B′ is conjugated to the surface of the second protein along a second plane in space.

7. (canceled)

8. The hierarchical protein structure of claim 1, wherein the one or more polynucleotides of the patch A are in about the same plane as the one or more polynucleotides of the patch B, and/or wherein the one or more polynucleotides of the patch A′ are in about the same plane as the one or more polynucleotides of the patch B′.

9. (canceled)

10. The hierarchical protein structure of claim 1, wherein the one or more polynucleotides of patch A are orthogonal to the one or more polynucleotides of the patch B, and/or wherein the one or more polynucleotides of the patch A′ are orthogonal to the one or more polynucleotides of the patch B′.

11.-13. (canceled)

14. The hierarchical protein structure of claim 1, wherein the one or more polynucleotides of the patch A and the one or more polynucleotides of the patch B comprises DNA, RNA, or a combination thereof, and/or wherein the one or more polynucleotides of the patch A′ and each of the one or more polynucleotides of the patch B′ comprises DNA, RNA, or a combination thereof.

15. (canceled)

16. The hierarchical protein structure of claim 1, wherein the one or more polynucleotides of the patch A have a different melting temperature than the one or more polynucleotides of the patch B, and/or wherein the one or more polynucleotides of the patch A′ have a different melting temperature than the one or more polynucleotides of the patch B′.

17.-19. (canceled)

20. The hierarchical protein structure of claim 1, wherein:
(i) the patch A comprises a plurality of polynucleotides and each of the plurality of polynucleotides has the same nucleic acid sequence, or the patch A comprises a plurality of polynucleotides and at least two polynucleotides contained within the plurality of polynucleotides have different nucleic acid sequences; and/or
(ii) the patch B comprises a plurality of polynucleotides and each of the plurality of polynucleotides has the same nucleic acid sequence, or the patch B comprises a plurality of polynucleotides and at least two polynucleotides contained within the plurality of polynucleotides have different nucleic acid sequences; and/or
(iii) the patch A′ comprises a plurality of polynucleotides and each of the plurality of polynucleotides has the same nucleic acid sequence, or the patch A′ comprises a plurality of polynucleotides and at least two polynucleotides contained within the plurality of polynucleotides have different nucleic acid sequences; and/or
(iv) the patch B′ comprises a plurality of polynucleotides and each of the plurality of polynucleotides has the same nucleic acid sequence, or the patch B′ comprises a plurality of polynucleotides and at least two polynucleotides contained within the plurality of polynucleotides have different nucleic acid sequences.

21.-27. (canceled)

28. The hierarchical protein structure of claim 1, wherein the first protein and the second protein are different.

29. (canceled)

30. The hierarchical protein structure of claim 1, further comprising a third protein comprising a patch B′ comprising one or more polynucleotides conjugated to the surface of the third protein which hybridizes to the patch B of the first protein or the patch B′ of the second protein.

31. A method of making a hierarchical protein structure comprising contacting:
(a) a first protein comprising: (i) a patch A comprising one or more polynucleotides conjugated to the surface of the first protein; and (ii) a patch B comprising one or more polynucleotides conjugated to the surface of the first protein; and
(b) a second protein comprising: (i) a patch A′ comprising one or more polynucleotides conjugated to the surface of the second protein; and (ii) a patch B′ comprising one or more polynucleotides conjugated to the surface of the second protein;
wherein the one or more polynucleotides of the patch A is sufficiently complementary to the one or more polynucleotides of the patch A′ to hybridize, and wherein the contacting is performed under conditions that result in the one or more polynucleotides of the patch A hybridizing to the one or more polynucleotides of the patch A′,
thereby making the hierarchical protein structure.

32. The method of claim 31, wherein the one or more polynucleotides of the patch B is sufficiently complementary to the one or more polynucleotides of the patch B′ to hybridize under said conditions.

33. (canceled)

34. The method of claim 31, further comprising hybridizing the one or more polynucleotides of the patch B to the one or more polynucleotides of the patch B′.

35.-43. (canceled)

44. The method of claim 31, wherein:
(i) each of the one or more polynucleotides of the patch A is conjugated to an amino acid residue of the first protein; and/or
(ii) each of the one or more polynucleotides of the patch B is conjugated to an amino acid residue of the first protein; and/or
(iii) each of the one or more polynucleotides of the patch A′ is conjugated to an amino acid residue of the second protein; and/or
(iv) each of the one or more polynucleotides of the patch B′ is conjugated to an amino acid residue of the second protein.

45.-48. (canceled)

49. The method of claim 31, wherein the first plane of the first protein and the first plane of the second protein, and the second plane of the first protein and the second plane of the second protein, comprise different amino acid residues that allow for orthogonal conjugation of different polynucleotides along the first plane of the first protein and the first plane of the second protein relative to polynucleotides along the second plane of the first protein and the second plane of the second protein.

50.-64. (canceled)

65. The method of claim 31, wherein the one or more polynucleotides of the patch A hybridizes to the one or more polynucleotides of the patch A′ before the one or more polynucleotides of the patch B hybridizes to the one or more polynucleotides of the patch B′.

66.-69. (canceled)

70. The method of claim 65, wherein:
(i) the one or more polynucleotides of the patch A are complementary to the one or more polynucleotides of the patch A′; and/or
(ii) the one or more polynucleotides of the patch A are complementary to the one or more polynucleotides of the patch A of another protein; and/or
(iii) the one or more polynucleotides of the patch B are complementary to the one or more polynucleotides of the patch B′; and/or
(iv) the one or more polynucleotides of the patch B are complementary to the one or more polynucleotides of the patch B of another protein.

71.-75. (canceled)

76. The method of claim 31, wherein hybridization of the one or more polynucleotides of the patch A to the one or more polynucleotides of the patch A′ enables hybridization of the one or more polynucleotides of the patch B to the one or more polynucleotides of the patch B′.

77.-78. (canceled)
